(19) United States
(12) Patent Application Publication
(10) Pub. No.: US 2026/0153498 A1
(43) Pub. Date: Jun. 4, 2026

(54) VCR-SEEK

(71) Applicant: THE UAB RESEARCH FOUNDATION, BIRMINGHAM, AL (US)

(72) Inventors: Kevin Maroney, Birmingham, AL (US); Anju Bansal, Vestavia Hills, AL (US); Paul Andres Goepfert, Birmingham, AL (US)

(21) Appl. No.: 19/406,122

(22) Filed: Dec. 2, 2025

Related U.S. Application Data
(60) Provisional application No. 63/727,506, filed on Dec. 3, 2024.

(51) Int. Cl. G01N 33/50; G01N 33/68

(52) U.S. Cl. CPC G01N 33/505; G01N 33/5023; G01N 33/6845

(57) ABSTRACT

Disclosed herein is a pipeline to identify variable chains (VCR), which encompass T cell receptors (TCRs) and B cell receptors (BCRs), that recognize epitopes in their low affinity using a bioinformatic statistical method with 10× data that encompasses both preprocessing and identification of antigen-specific VCRs. Also disclosed are methods for treating subjects having viral infections or cancer, the methods comprising administering antibodies from B cells transduced with antigen-specific BCRs and/or modified allogenic T cells transduced with antigen-specific TCRs identified through use of the pipeline.

### CROSS-REFERENCE TO RELATED APPLICATIONS

This application claims the benefit of U.S. Provisional Application No. 63/727,506 filed on Dec. 3, 2024, which is incorporated herein by reference in its entirety.

### STATEMENT OF GOVERNMENT INTEREST

This invention was made with Government Support under Grant No. AI 162168 awarded by the National Institutes of Health. The Government has certain rights in the invention.

### BACKGROUND

The seminal work demonstrating that vaccine-induced Mamu-E restricted CD8 T cells are able to prevent the establishment of SIV has generated a great deal of interest in this research area in the context of HIV (Hansen, S. G. et al. Sci. Immunol. 2022 7:eabn9301; Hansen, S. G. et al. Science 2016 351:714-720). Nonclassical restriction of CD8 T cells could be especially important for vaccines, as only 2 human leukocyte antigen (HLA)-E alleles, present at ˜50% each across humans need to be considered (Hansen, S. G. et al. Sci. Immunol. 2022 7:eabn9301; Hansen, S. G. et al. Science 2016 351:714-720; Voogd, L. et al. Trends Immunol. 2022 43:355-365; Sharpe, H. R. et al. Clin. Exp. Immunol. 2019 196:167-177). The immunodominant HIV-1 peptide KF11 (KAFSPEVIPMF, Gag) as well as subdominant KL9 (KALGPAATL, Gag [335-343]), both previously described as being restricted by controller-associated HLA-B*57 (HLA-Ia family), have also been shown to be HLA-E (HLA-Ib family) restricted (KF11-E-CD8s) (Bansal, A. et al. J. Clin. Invest. 2021 131:148979). These findings were complemented by another study which described the peptide RL9 (RMYSPTSIL, Gag) as also being HLA-E restricted (RL9-E-CD8s), consistent with the possibility that HLA-E may present multiple HIV-1 epitopes recognized by human CD8+ T cells (Yang, H. et al. Sci. Immunol. 2021 6:eabg1703). However, HLA-E restricted CD8 T cells remain relatively understudied especially in the context of HIV infection.

Despite the encouraging results observed in rhesus macaques in the context of SIV infection and MHC-E-restricted CD8s, identifying HLA-E-restricted epitopes which are recognized by E-CD8s in humans has been hampered by their extremely low affinity in HIV (Yang, H. et al. Sci. Immunol. 2021 6:eabg1703; Wallace, Z. et al. Mol. Ther. J. Am. Soc. Gene Ther. 2024 S1525-0016(24)00010-8; He, W. et al. J. Exp. Med. 2023 220:e20221941). This largely necessitates novel approaches to either alter the natural sequence and/or conformation of HLA-E/HIV peptide complex to artificially engineer a stable peptide-HLAE (pHLA-E) (Yang, H. et al. Sci. Immunol. 2021 6:eabg1703). It has been recently shown through HLA-E monomer peptide exchange that even at molar excess (100 μM), the highest affinity HIV-1 peptides have nowhere near the affinity of Mtb-derived peptides (Walters, L. C. et al. Eur. J. Immunol. 2020 50:2075-2091). Why HIV-I specifically has such low affinity HLA-E-bound peptides as compared to M. tuberculosis (Mtb) or human cytomegalovirus (HCMV) is unclear. However, HLA-E restricted HIV-1 peptides are still immunogenic in humans, highlighting their potential importance.

### SUMMARY

Disclosed herein is a method for identifying a variable chain receptor (VCR) family/clonotype subset for an antigen, the method including at least the steps of (a) isolating at least one type of lymphocyte from a plurality of subjects having a disease associated with the antigen, where the plurality of subjects are classified into two or more disease conditions based on detectable levels of the antigen in the plurality of subjects; (b) culturing the at least one type of lymphocyte with a plurality of disease-appropriate cell lines to create cell populations representing each of the two or more disease conditions, to provide unstimulated cells for each of the two or more disease conditions; (c) culturing the at least one type of lymphocyte with the plurality of disease-appropriate cell lines loaded with antigen to provide stimulated conditions for each of the two or more disease conditions; (d) conducting single-cell RNA sequencing (scRNA-seq) and single-cell TCR sequencing (scTCR-seq) or single-cell BCR sequencing (scBCR-seq) on each of lymphocyte from steps (b) and (c) to identify the count and sequence of VCRs in each lymphocyte across all conditions; (e) identifying families or clonotypes for lymphocytes that had a statistically significant change in count and frequency between stimulated and unstimulated conditions; and (f) comparing each of the two or more disease conditions to identify antigen-specific family/clonotype subsets; and (g) cloning identified VCRs into a cell based reporter system to test whether, in the presence of the specific antigen, disease condition, relevant stimulant, or any combination thereof, the VCRs demonstrate functional avidity.

This pipeline, combined with metadata information for the source of every and all single TCR α, TCR β or paired TCR α/β families and clonotypes identifies ALL significantly antigen-specific TCRs between all no antigen (unstimulated) to with antigen (stimulated) CD8s for every classification and sub-classification (e.g., count/frequency of queried single or paired family or clonotype within all conditions, within all of one condition (all controller conditions, all of a given HLA-X conditions), or within all sub-classification (within all controller-B57, all PBMC-elite controller, etc. conditions)). These specific metadata will change with the groupings and metadata given for a given dataset. Likewise, this pipeline works with antigen-specific BCRs, which can be transduced into B cells from which antibodies can be produced.

A script applying this method can be used for a single comparison for a given antigen (unstimulated) to with antigen (stimulated), but is also robust enough to identify antigen-specific CD8s between any 2 metadata, whether they be specific single metadata, or a combination of subclassifications of metadata (for example, those which increase significantly with antigen in all samples (using TCR counts to compare unstimulated to stimulated across ALL metadata groups) or within only males, or within only males who smoke, etc. dependent on metadata).

In some embodiments, the method further involves immunoassaying CD8+ T cells identified in step (e) for expression levels of one or more cytokines and growth factors selected from the group consisting of EGF, CCL11, G-CSF, GM-CSF, IFNα2, IFNγ, IL-1α, IL-1β, IL-1RA, IL-2, IL-3, IL-4, IL-5, IL-6, IL-7, IL-8, IL-10, IL-12 (p40), IL-12 (p70), IL-13, IL-15, IL-17A, IL-17E, IL-17F, IL-18, IL-22, IP-10, MCP-1, M-CSF, MIG, MIP-1α, MIP-1β, PDGF-AA, PDGF-AB/BB, RANTES, TNFα, LTA, VEGF-A. For example, this immunoassay can be a high-parameter Luminex assay.

Also disclosed is a method of treating a subject with a pathogen or cancer, comprising administering to a subject a modified allogeneic T cell transduced with antigen-specific T cell receptors (TCRs) associated with positive disease outcome identified through the disclosed method or administering an antibody from a modified B cell transduced with an antigen specific BCR.

Also disclosed is a method of developing a database (which will increase in sensitivity and breadth as more sequenced VCRs are added of TCRs/BCRs associated with a positive disease outcome which may be queried by vaccine trials to determine if in a given vaccination cohort TCRs and/or BCRs are preferentially inducing antigen-specific variable chain receptors (VCRs, including TCRs and BCRs) associated with positive disease outcome. They can also be queried by health care professionals to determine if in a given patient, the proper antigen-specific VCRs associated with positive disease outcome are being induced.

The disclosed method can also be used to identify scientific reagents, wherein identified most highly antigen-specific BCRs (antibodies) may be used in conjunction with fluorophores to generate high-specificity flow cytometry, western blotting, or other antibody-involved immuno- or other assays.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A and 1B contain Luminex data showing distinct functionality of E-versus B57-CD8s. All 8 HLA-class I restricted responses were obtained to either no peptide or KF11 across all responders. All concentrations for each donor in each condition were normalized to Parental-cell line KF11 response (41A3.CD4 “null” cell line pulsed with KF11 and co-cultured with CD8s from all 8 donors) individually except for PBMC response. Determinant analysis for clustering by HLA, Control, or by HLA was done within control subsets of all samples utilizing all cytokine concentrations. Individual cytokines which significantly increase from DMSO to KF11 conditions and/or showed a significant difference between HLA conditions by 2-way ANOVA from within each HLA were grouped by cytokines which are HLA-E high (FIG. 1A) or HLA-B57 high (FIG. 1B). Paired t test and 2-way ANOVA significance values are indicated as shown (*, **, ***, **** represent p<0.05, 0.01, 0.001, and 0.0001 respectively).

FIG. 2 shows substantial HLA-I cross-restriction of antigen-specific E01-, E03-, and B57-CD8s. Pyramid plot representing total proportion of all TCR Vαβ family pairs observed in response to HLA-B*57:01-, HLA-E*01:01-, and HLA-E*01:03-restricted KF11. Those pairs which were observed in 2 or 3 HLA restriction states are indicated as Dual- or Tri-restricted with an arrow and bracket.

FIG. 3 shows functional affinity of TRAV5-containing CDR3 pairs cloned into a Jurkat reporter cell line and co-incubated with HLA-B*57:01 or HLA-E*01:01-transduced HLA-null line pulsed with KF11.

FIGS. 4A to 4C show multiply-restricted CD8s exhibit signatures of higher metabolic activity. FIG. 4A shows PCA of highest proportion CD8s in single, dual, and tri-restricted metaclonotype clusters of FIG. 3, colored by membership. FIG. 4B shows gene ontology (GO) bubblemap of CD8s, with relative percentage of enriched genes contributing to each GO node represented as a pie chart. Colors map to “Enriched set” of figure legend in each pairwise comparison indicated under “Comparison.” Analysis performed using Metascape. FIG. 4C shows relative enrichment for each enriched set of multiply-vs singly-restricted comparison for the indicated GO terms as bubblemap. Size of circles correlates with confidence and significance of the enriched set as measured by Abs(logQ) value to the gene ontology term. This is based on both the number and types of genes included in the gene list of the indicated GO term, the number indicated by color as per legend.

FIG. 5 is a Violin plot of top variable cytokines across dataset separated by cluster.

FIGS. 6A to 6E show identified antigen-specific clonotypes are cross-restricted. Clonotypes identified as being cross-restricted in 10× experiment through described bioinformatics pipeline were cloned into a Jurkat cell line with an NFAT-luciferase reporter. Transformed cells were then co-cultured with 41A3.CD4.B57, 41A3.CD4.E01, or 41A3.CD4.E03 cell lines loaded with KF11 and fold change (FC) of luciferase signal over co-culture with peptide on irrelevant HLA-A*02 expressing cell line is shown. FIG. 6A shows the fold change of all tested clonotypes is shown as a line graph, with the B*57:01, E*01:01, or E*01:03 co-culture signal FC of the same clonotype indicated by blue, red, or orange dots respectively. The mean FC of all tested clonotypes in the given graphs is shown below the respective condition (FIG. 6B) and represented as a bar graph for all tested clonotypes split by the control status where the clonotype was found to be statistically antigen-specific through the original bioinformatics pipeline. FIG. 6A was then broken down by control status for the noncontroller (FIG. 6C), controller (FIG. 6D), and elite controller (FIG. 6E). The top 1-3 clonotypes for every grouping are indicated next to the B57 dot of the indicated clonotype (“Gp . . . ”) for later reference.

FIGS. 7A and 7B show IL15 vaccine response signature observed in E03_B57 elite controller cluster. FIG. 7A is a Bubbleplot showing top gene ontology enrichment terms first unique to given cluster organized by cluster, then top terms shared by all clusters, colored by marker gene count and sized by FDR (abs log (Q)) value of term. FIG. 7B is a violin plot of top genes both within interleukin-15 signaling pathway and previously identified in macaques which prevent establishment of SIV infection. Significance consistent with rest of figures (*, **, ***, **** are p<0.05, 0.01, 0.001, 0.0001 respectively).

FIGS. 8A-8E show several Secreted Cytokines Confirmed to be Expressed within HLA-restricted CD8s. Violin plot of RNA expression for those cytokines identified through high-parameter Luminex in HLA-restricted co-culture within 10× Seurat clusters. HLA-E-restricted (FIG. 8A) or B57-restricted (FIG. 8B) cytokines which are significantly increasing compared to all other clusters by MAST are boxed in red or blue respectively. Relevant clusters are marked as E-CD8s or B57-CD8s below depending on relative number of cells for HLA-restricted metadata in those clusters (FIGS. 8C-8E). Exact p values are given when given cytokine is positive in more than 1 cluster and in both an E-CD8 and B57-CD8 cluster for comparison. All p values given are FDR-corrected (p=0.05, 0.01, 0.001, 0.0001 represented as *, **, ***, **** respectively).

FIGS. 9A to 9B the gating strategy and response magnitude for CD3-low HLA-restricted responses. FIG. 9A is a gating strategy for identifying CD3-low CD8+ T cells. FIG. 9B shows response magnitude of dually activated (CD69+ CD137+) CD3-low high avidity HLA-restricted CD8+ T cells to KF11 in each HLA group.

FIGS. 10A-10B show antigen specific and single cell TCR sequencing of CD8+ T cells obtained from 8 PLW show that TCRs which contain CDR3 α chains belonging to the TRAV5*01_TRBV*19 family can cross-recognize KF11 when presented by HLA-B*57:01, E*01:01, and E*01:03 primarily in controllers and elite controllers, whereas a majority of CD8+ T cells from non-controllers recognize KF11 primarily restricted by HLA-B*57 (TRAV14/DV4-TRBV2). FIG. 10C is a graph showing magnitude of CD69+ CD137+ response shown in FIG. 9B. FIG. 10D is a bar graph showing response frequency among noncontrollers, controllers, and elite controllers in cohort relative to HLA-B*57:01-, E*01:01-, and E*01:03-restricted KF11 from FIG. 9B.

FIG. 11 shows antigen-specific HLA-restricted clonotypes are transcriptionally distinct from one another. Heatmap of number cells with paired TCR clonotype sequence metadata found within each Seurat cluster within the ag-specific subset of 10× data.

FIGS. 12A-12B show antigen-specific B57- and E-CD8s are from the same memory subsets. Utilizing the CITE-seq CD45RA as well as CCR7 RNA expression levels within the antigen-specific CD8 subset, cells were assigned CD45RA+/− and CCR7+/− based on percentile expression level (data available on request). FIG. 12A is a heatmap of metadata for the number of cells assigned one of 4 memory subsets based on these positivity criteria (CCR7− CD45RA−, CCR7+ CD45RA−, CCR7− CD45RA+, CCR7+ CD45RA+ assigned effector memory (EM), central memory (CM), terminal effector memory re-expressing RA (TEMRA), and naïve (N) respectively), colored by Z-score. FIG. 12B is a bar graph representing number cells assigned each memory state in FIG. 12A separated by HLA-expression condition.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

In one aspect, disclosed herein is a method for identifying a variable chain receptor (VCR) family/clonotype subset for an antigen, the method including at least the steps of:
(a) isolating at least one type of lymphocyte from a plurality of subjects having a disease associated with the antigen, where the plurality of subjects are classified into two or more disease conditions based on detectable levels of the antigen in the plurality of subjects;
(b) culturing the at least one type of lymphocyte with a plurality of disease-appropriate cell lines to create cell populations representing each of the two or more disease conditions, to provide unstimulated cells for each of the two or more disease conditions;
(c) culturing the at least one type of lymphocyte with the plurality of disease-appropriate cell lines loaded with antigen to provide stimulated conditions for each of the two or more disease conditions;
(d) conducting single-cell RNA sequencing (scRNA-seq) and single-cell TCR sequencing (scTCR-seq) or single-cell BCR sequencing (scBCR-seq) on each of lymphocyte from steps (b) and (c) to identify the count and sequence of VCRs in each lymphocyte across all conditions;
(e) identifying families or clonotypes for lymphocytes that had a statistically significant change in count and frequency between stimulated and unstimulated conditions; and
(f) comparing each of the two or more disease conditions to identify antigen-specific family/clonotype subsets; and
(g) cloning identified VCRs into a cell based reporter system to test whether, in the presence of the specific antigen, disease condition, relevant stimulant, or any combination thereof, the VCRs demonstrate functional avidity.

In some aspects, the method can include additional optional steps. In a further aspect, following step (g), upon storing all VCRs in a matrix as associated with a given control state or positive disease outcome, vaccinee or drug VCRs for trial vaccines or drugs can be then be compared to determine overlap as a potential biomarker for “controller-like” or higher likelihood of positive disease outcome under that vaccine or drug-treatment condition, as an output of the analysis from step (g). In another optional aspect, following step (f), the family/clonotype subsets can be identified as antigen-specific and characterized in a bioinformatics pipeline for later functional analysis of antigen-specific subsets in relation to various disease states.

In a further aspect, in the disclosed method, the VCR can be a T cell receptor (TCR), the at least one type of leukocyte can be a CD8+ T cell, the two or more disease conditions can be two or more human leukocyte antigen (HLA) conditions, the family/clonotype subset can be an HLA-TCR family/clonotype subset, the plurality of disease-appropriate cell lines can be HLA-expressing cell lines, scRNA-seq and scTCR-seq are conducted in step (d), the family/clonotype subset can include TCR α, β, paired αβ families or clonotypes, and the cell based reporter system can be a Nuclear Factor of Activated T-cells (NFAT)-reporter system.

In an alternative aspect, in the disclosed method, the VCR can be a B cell receptor (BCR), the at least one type of leukocyte can be a B cell, scRNA-seq and scBCR-seq are conducted in step (d), antigen-specific BCR heavy and light chain sequences are transfected into BCR-null expression systems, isolated, and purified; and the cell based reporter system can be selected from ELISA, multimer detection, surface plasmon resonance (SPR) or any combination thereof.

In some aspects, the method further includes immunoassaying CD8+ T cells identified in step (e) for expression levels of one or more cytokines and growth factors selected from the group consisting of EGF, CCL11, G-CSF, GM-CSF, IFNα2, IFNγ, IL-1α, IL-1β, IL-1RA, IL-2, IL-3, IL-4, IL-5, IL-6, IL-7, IL-8, IL-10, IL-12 (p40), IL-12 (p70), IL-13, IL-15, IL-17A, IL-17E, IL-17F, IL-18, IL-22, IP-10, MCP-1, M-CSF, MIG, MIP-1α, MIP-1β, PDGF-AA, PDGF-AB/BB, RANTES, TNFα, LTA, VEGF-A. In an aspect, the immunoassay can be a high-parameter Luminex assay, but other assays are contemplated and should be considered disclosed. Further in this aspect, the method can include conducting a CITE-seq (cellular indexing of transcriptomes and epitopes) assay on the CD8+ T cells identified in step (e). In a still further aspect, in the disclosed method, step (f) includes identifying HLA-restricted TCR family/clonotype subsets that are the most antigen-specific with the highest difference in unstimulated to stimulated conditions.

In another aspect, the method is general and can be used for either TCRs or BCRs, viruses other than or in addition to HIV, antigen specific conditions other than or in addition to HLA, and the like. Further in this aspect, examples relating to HIV are exemplary and are not intended to be limiting.

In one non-limiting aspect, the antigen can be a viral antigen such as, for example, a human immunodeficiency virus (HIV) antigen and step (a) includes obtaining CD8+ T cells from each of i) an ART-naïve elite controller (EC) subject, ii) an ART-naïve controller (C) subject, and an ART-naïve noncontroller (NC) subject. Further in this aspect, the EC subject has a plasma viral load less than 200, the C subject has a plasma viral load from 20 to 2000, and the NC subject has a plasma viral load greater than 2000.

Also disclosed herein is an antigen-specific TCR associated with a positive disease outcome identified through the disclosed method. In another aspect, disclosed herein is a modified allogenic T cell transduced with the antigen-specific TCR. In a still further aspect, disclosed herein is a method of treating a subject having a viral infection or cancer, the method including administering to a subject the modified allogeneic T cell.

In another aspect, disclosed herein is a B cell transduced with the antigen-specific BCR identified through the disclosed method. In another aspect, disclosed herein is an antibody produced by the B cell. In still another aspect, disclosed herein is method of treating a subject having a viral infection or cancer, the method including administering to a subject the antigen-specific antibody.

In one aspect, disclosed herein is a database of VCRs associated with positive disease outcome produced by conducting the disclosed method a plurality of times and storing data obtained therefrom in a computer readable medium. Further in this aspect, disclosed herein is a method for determining effectiveness of a vaccine or drug treatment against a disease, wherein VCRs associated with positive disease outcome are present in the disclosed database, the method including administering the vaccine or drug treatment to a plurality of subjects and comparing data from samples from the subjects to the database to determine presence or absence of the VCRs associated with positive disease outcome in the subjects, where presence of the VCRs indicates the vaccine or drug treatment is effective. Also disclosed herein is a method for determining effectiveness of a treatment for a disease in a subject, wherein VCRs associated with positive disease outcome are present in the disclosed database, the method including comparing data from a sample the subject to the database to determine presence or absence of the VCRs associated with positive disease outcome in the subject, where presence of the VCRs indicates the treatment is effective.

In one aspect, disclosed herein is a reagent for use in an immunoassay, the reagent being an antibody as disclosed herein, wherein the antibody is conjugated to a fluorophore. In an alternative aspect, the disclosed antibodies can, with or without labels, be used for other research purposes.

### Definitions

It must be noted that, as used in the specification and the appended claims, the singular forms “a,” “an,” and “the” include plural referents unless the context clearly dictates otherwise.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: CD8 T Cell Clonotypes Recognize HIV-1 Cross-Restricted by HLA-E*01:01/03 and HLA-B*57:01 with Different Functional Profiles

### Introduction

To elucidate the role that E- and B57-CD8s play in the chronic HIV-1 infection, we utilized targeted scRNA/TCR-sequencing and an unbiased high-parameter Luminex assay to identify factors which are shared or unique between the two HLA class I alleles (HLA-I). We determined that KF11-CD8s have the potential to be dually restricted by both B57- and E-CD8s (E*01:01 and/or E*01:03). Dual restriction was biologically confirmed when these clonotypes were tested using an in vitro based functional assay (Anmole, G. et al. J. Immunol. Methods 2015 426:104-113). Despite this cross-restriction, B57- and E-CD8s were found to be functionally distinct in their secretory and RNA expression profile. Interestingly, we identified a cluster of dually-restricted clones that significantly increased in an elite controller who exhibited an IL15-response signature associated with prevention of infection in SIV vaccinated and challenged macaques (Barrenäs, F. et al. PLoS Pathog. 2021 17:e1009278).

### Results

### Study Design

CD8+ T cells of people with HIV (PWH) were mapped for KF11 responses in an IFNγ ELISPOT assay. Those n=8 (2 elite controllers (EC), 4 controllers (C), and 2 noncontrollers (NC), with plasma viral load <200, <2000, or >2000 respectively) with HIV-1 KF11 responses were subjected to SMART-seq2 based scRNA-seq and scTCR-seq as well as a high-parameter Luminex assay upon co-culture with KF11-loaded single HLA-B*57,-E*01:01, and -E*01:03 expressing cell lines. A subset of samples (one each of C, EC and NC from these 8 PWH were then subjected to 10× sequencing to identify HLA-restricted correlates of only their most antigen-specific clonotype subset.

### Cytokine Profiles of B57-and E01/3-CD8s are Functionally Distinct

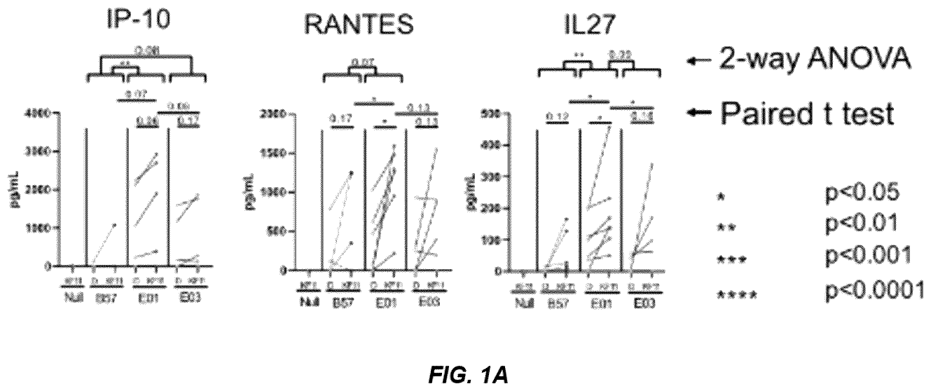

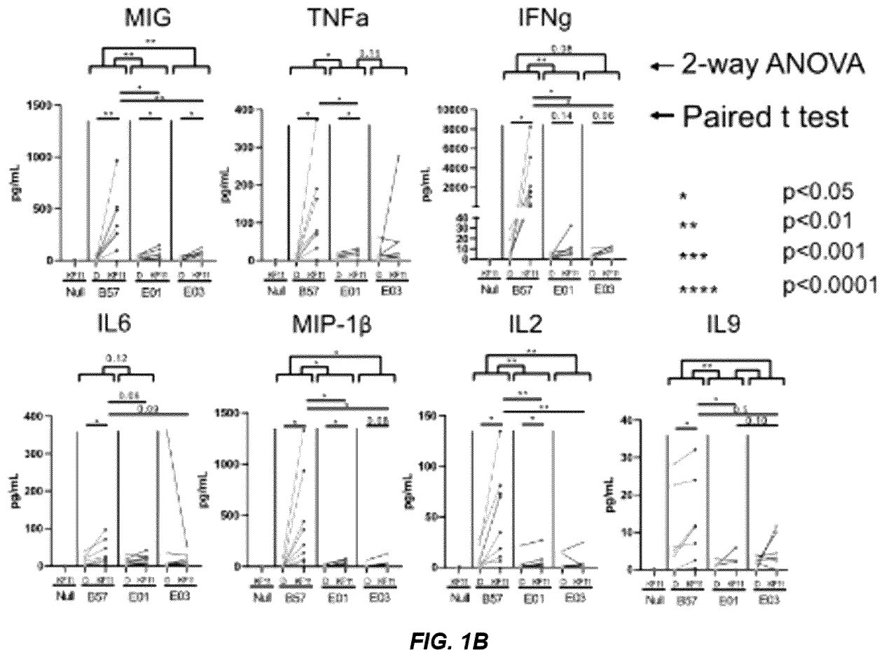

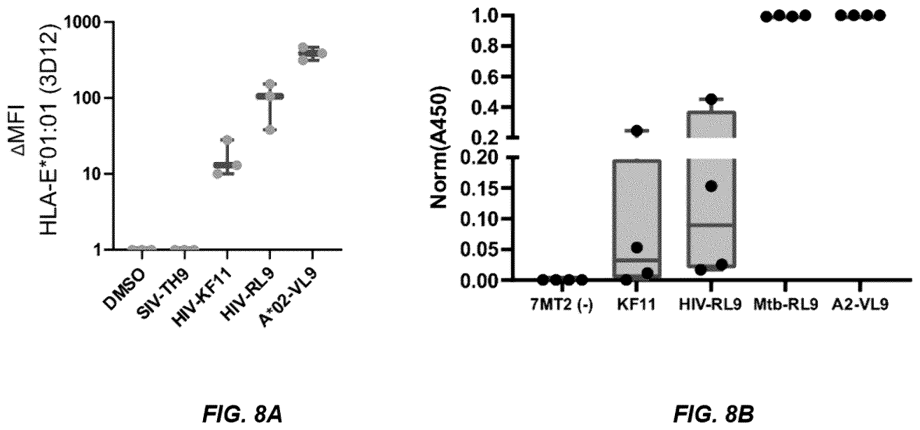

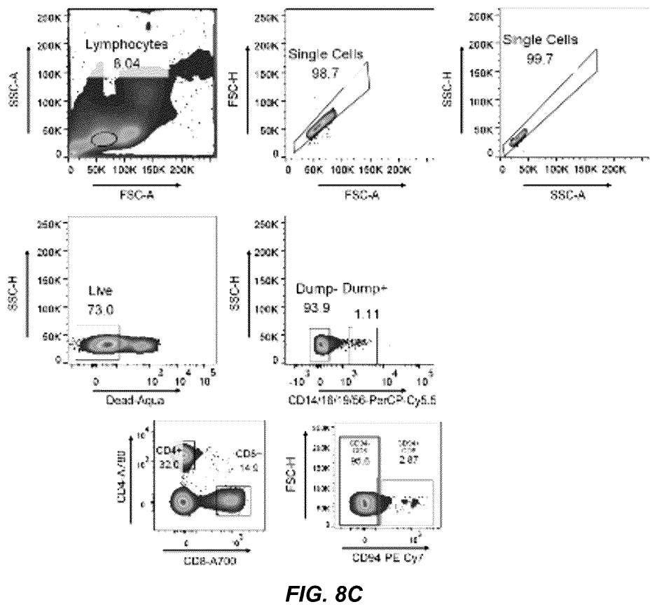

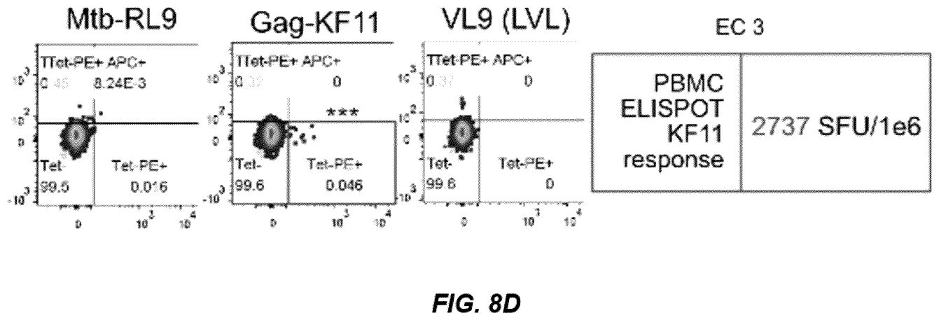

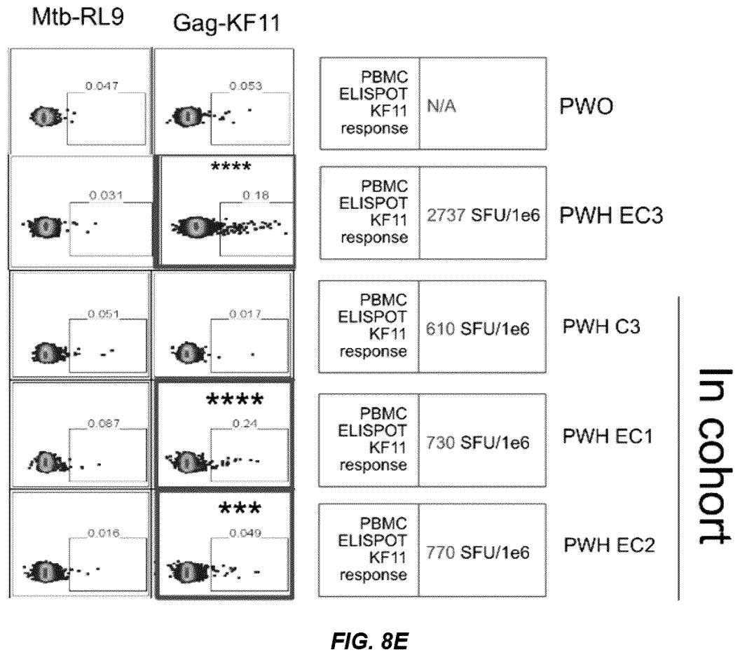

We utilized an unbiased 38 plex human cytokine/chemokine panel to compare the functionality of E*01:01/03 and B*57-CD8s. The cytokines which we screened were assessed for their increase upon antigen stimulation (from DMSO to KF11 pulse by paired T test) as well as for their HLA-restricted specificity (2-way ANOVA performed between HLA-E*01:01 or HLA-E*01:03 and HLA-B*57 DMSO and KF11 conditions). HLA-restricted (B57, E01, E03, Null/Parental) secreted proteomes cluster away from one another and are thereby functionally distinct by 3 component groupings of cytokines plotted. Additionally, these responses are functionally distinct when clustered by control status on the same 2 component groupings. When clustered by HLA response but subset by noncontroller status, HLA-B*57-restricted responses seem to drive the majority group-derived variation, whereas in the 4 controllers and 2 elite controllers B57, E01, and E03-restricted responses form spatially distinct clusters. Of the cytokines examined, CXCL10 (IP-10), IL27, and CCL5 (RANTES) were increased significantly in E-CD8s (FIG. 1A). In contrast, CXCL9 (MIG), TNFα, IFN-γ, IL6, CCL4 (MIP-1β), IL2, and IL9 were increase significantly increased in B57-CD8s (FIG. 1B). IP-10 demonstrated an increased trend post HLA-E*01:01-restricted response (p=0.06). Several cytokines (TNFα, IFNγ, MIP-1β, IL2) demonstrated a significant increase post stimulation in the context of HLA-B*57 restriction, while only RANTES significantly increased in the context of HLA-E restriction. Two of three E-CD8 cytokines and three of 6 B57-CD8 cytokines were confirmed later to be transcribed in these CD8 subsets upon 10× analysis (FIGS. 8A-8B).

Single Cell TCR Sequencing (scTCR Seq) Shows that TCR Cross-Restriction is Driven Primarily by TRAV5*01

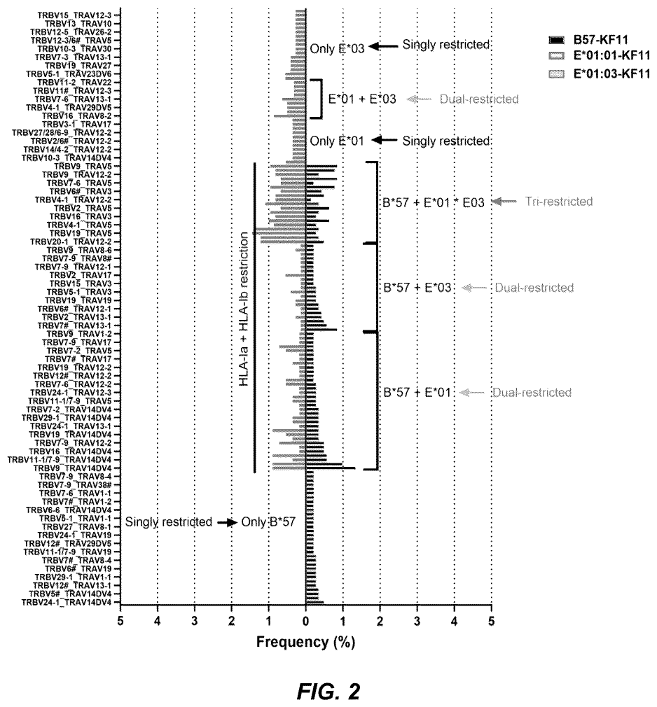

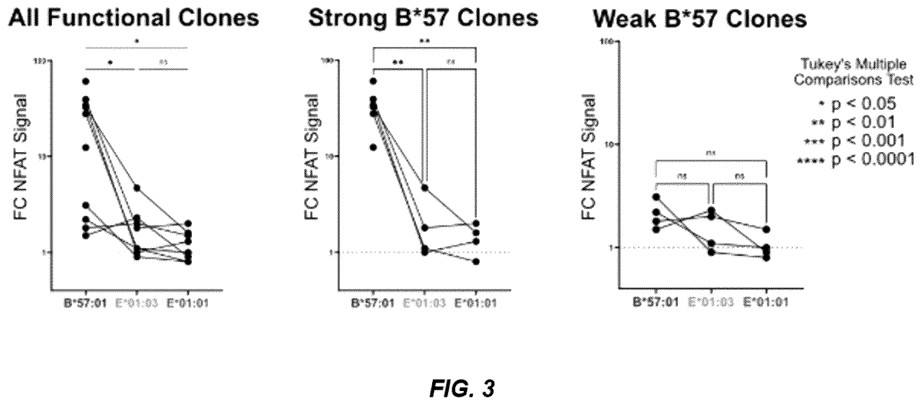

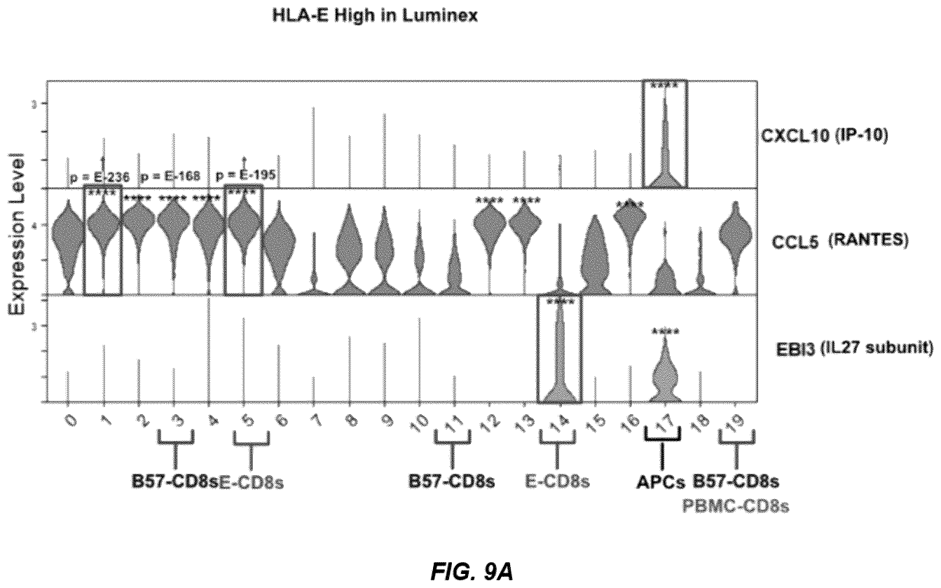

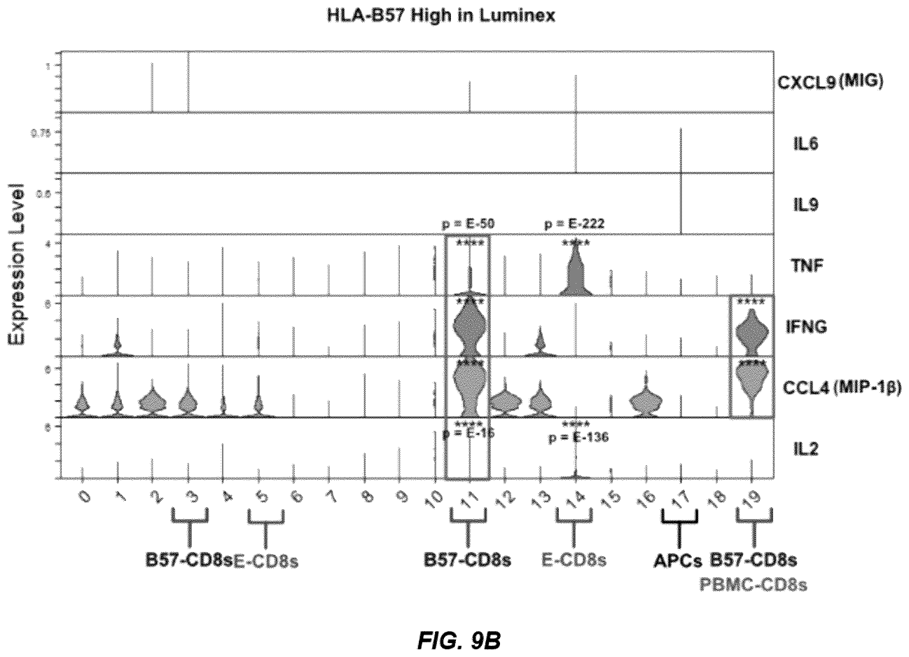

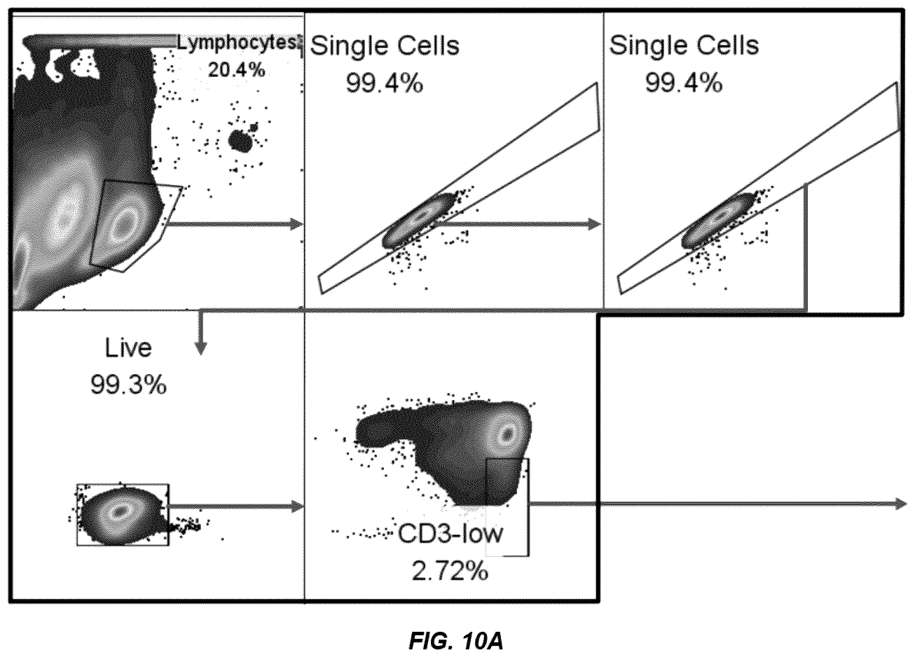

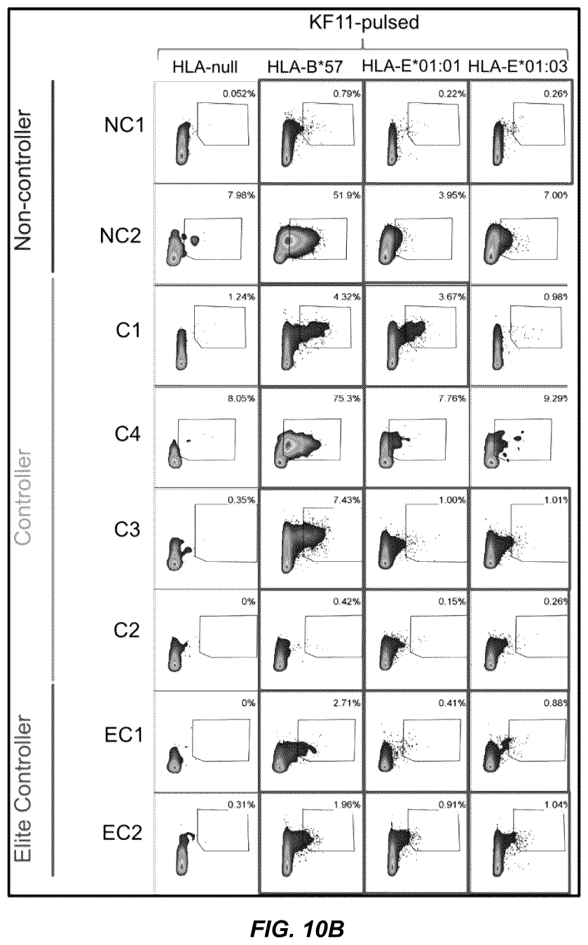

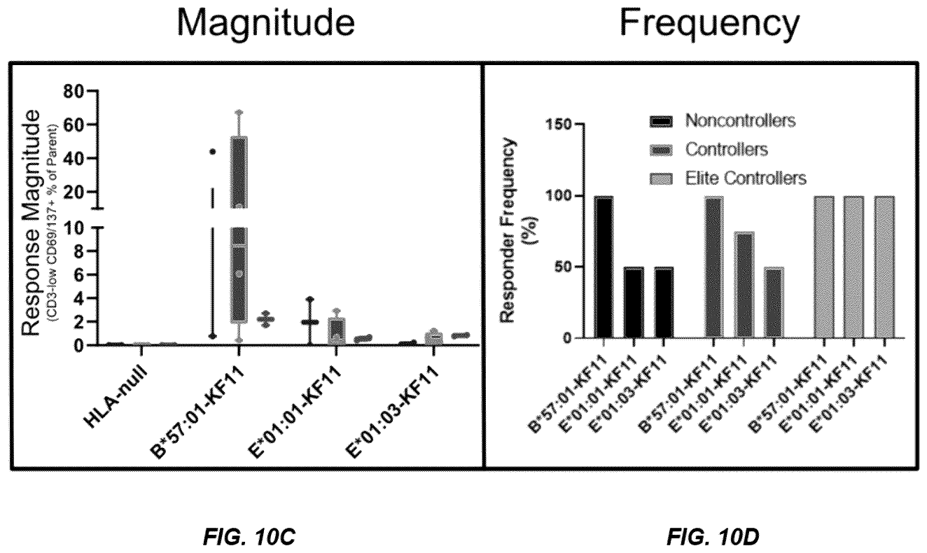

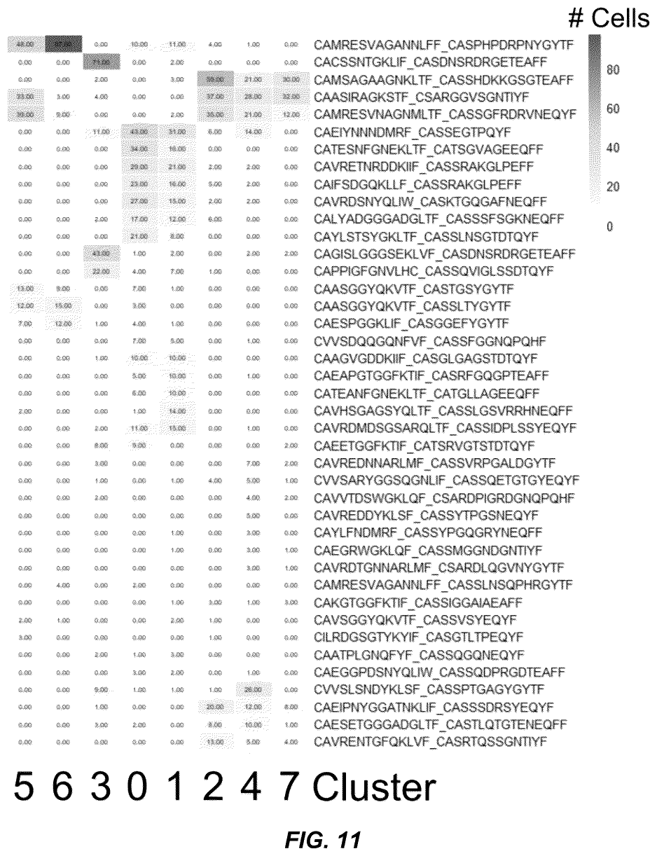

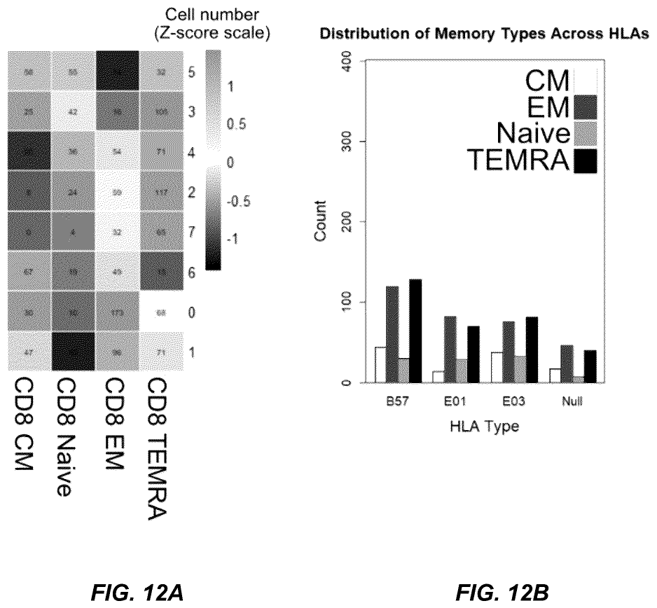

CD8+ T cells which were found to be activated and upregulating Activation Inducible Markers (AIM) CD69 and CD137 at a significantly higher level in response to stimulation with KF11 peptide in an HLA-restricted fashion over stimulation with KF11 in coincubation with the parental line were sorted for further analysis (FIGS. 9A-9B and 10C-10D). We performed scRNA/TCR-seq on the same N=8 patient samples (as in FIGS. 1A-1B) to complement the functional data obtained through the Luminex assays. KF11-specific (CD8+ CD3-dim) T cells were single cell sorted into a 96-well plate and sequenced. Across these 8 donor samples, the majority of responses were cross-restricted based on paired TCR families alone (FIG. 2). To account for the inherent heterogeneity in these highly variable TCR clonotype CDR3 sequences across multiple people (even specific for the same antigen) and the unlikelihood of finding a single public clonotype, we used TCRdist3 to identify clusters of highly related paired clones. From this analysis, we observed distinct singly-restricted clusters of HLA-E*01:03-restricted and HLA-B*57:01 restricted clonotypes, several dual-restricted HLA-E*01:01 and B*57:01, and a large cluster of tri-restricted clonotypes. Interestingly, the single feature in common with almost every clonotype in this cluster was the presence of a Vα CDR3 sequence belonging to the TRAV5 family, though there was also moderate enrichment for TRBV19 clonotypes. Additionally, the majority of clonotypes in this cluster were identified in controllers and elite controllers, while most clonotypes identified in noncontrollers mapped to a single cluster of mono-B*57:01-restricted clones. TRAV5-containing clones specific for KF11 were also confirmed to be restricted by both HLA-B57 and HLA-E*01:01 as well as HLA-E*01:03 as measured by NFAT signal fold change increase over KF11-pulsed HLA-A02 line control (FIG. 3). Interestingly, both E01- and E03-CD8s had significantly lower avidity (FC NFAT signal) for peptide-bound HLA (pHLA) than B57-CD8s.

### Cross-Restricted Clones Express Signatures of Higher Metabolic Activity

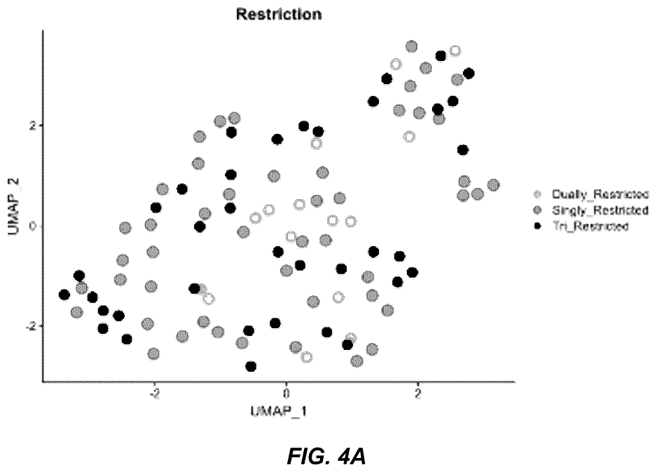

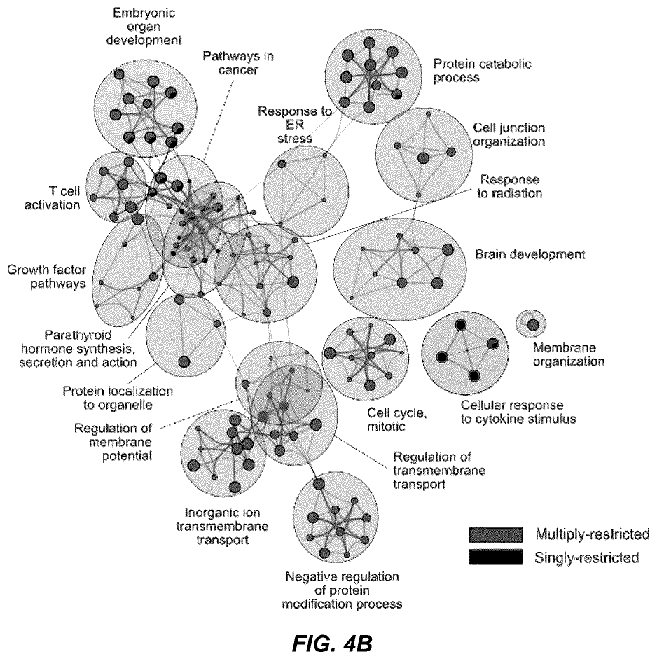

We next wanted to examine if transcriptional features distinguished this cross-restriction phenotype from the other clonotypes in this dataset. To this end, we linked the cluster metadata for only clones with a paired Vα and Vβ TCR in each tri-, dual-, or singly-restricted cluster to the scRNA-seq data obtained from this assay and performed MAST in a pairwise analysis (FIGS. 4A-4C). We identified several features shared by cross-restricted CD8s belonging to either the dually or tri-restricted (multiply restricted) CD8 metaclonotype clusters in comparison with singly restricted. At the bulk level, these clones did not appear significantly different from one another if clustered on a UMAP (FIG. 4A). However, if directly compared, multiply restricted clones significantly upregulate genes associated with transmembrane transport and protein catabolic activity, mitosis, and various metabolic pathways as well as T cell activation pathway genes in comparison to singly-restricted clones (FIGS. 4B, 4C).

### Single Cell Transcriptional Expression Clusters Distinctly Depending on HLA Restriction Status

Single cell 96 well based analyses represent data on a limited number of cells. Therefore, to examine if our observations are a general feature of a larger CD8+ T cell repertoire, we performed an in-depth analysis with 10× genomics based single cell immune profiling in 3 PWH (one each C, EC, and NC) based on sample availability. To examine the memory subsets and markers which may differentiate HLA-E restricted from B57-restricted CD8 T cell responses to HIV, we adopted a broader sorting strategy with more multiplexed conditions. We performed 10× scRNA-seq, TCR-seq, and CITE-seq on enriched activated and unactivated populations of both unstimulated (DMSO, no antigen) and stimulated (KF11-loaded) HLA-restricted conditions as well as PBMCs. Initial demultiplexing and pre-processing of the 10× data as well as initial quality control was carried out using Seurat as described in Methods section.

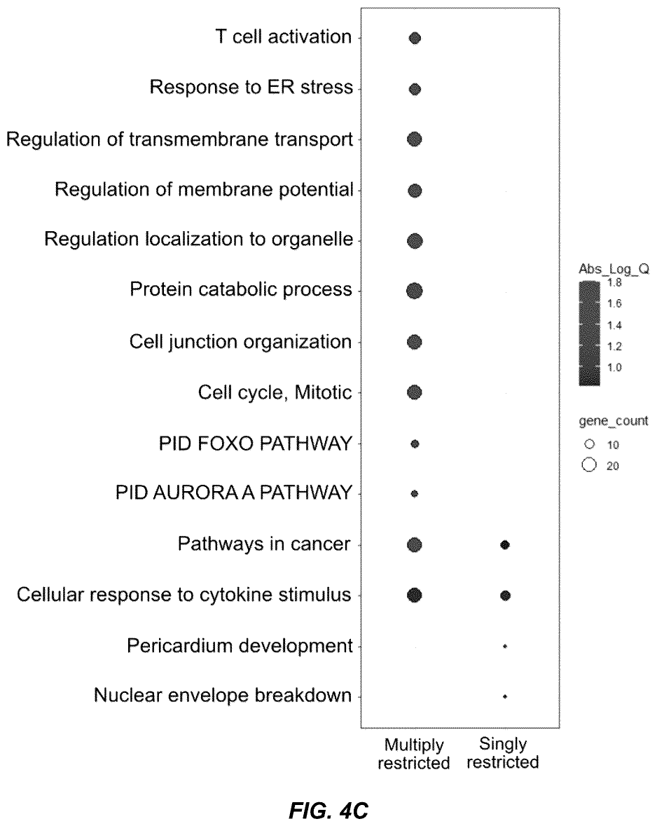

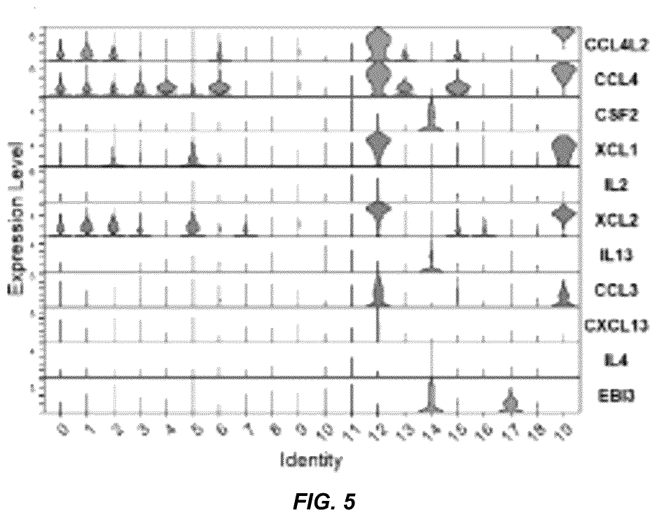

We initially determined that CITE-seq expression of memory markers CD45RA, CD45RO, RNA expression of CCR7 as well as CITE-seq expression of activation markers CD69, CD137, and LAMP1 were in distinct clusters suggesting that the memory and activation state of these clusters could later be called through metadata based on quantile expression. Additionally, Seurat UMAP clustering seemed to be mainly influenced by controller status but also by HLA restriction. The most variably expressed genes were predominantly immune-related and served to differentiate transcriptionally distinct clusters (FIG. 5). We lastly determined whether the transcriptomes of B57- and E-CD8s were as transcriptionally distinct as they were at the secreted proteome level. To this end, we performed a pairwise MAST analysis between B57-CD8s and grouped E01- and E03-CD8s. Within even this analysis which does not take into account differences between E01- and E03-CD8s, several of the same cytokines previously observed to be enriched in E- or B57-CD8s were confirmed (EBI3 as a subunit of IL27 was upregulated in E-CD8s, CCL4 and IFNG up in B57-CD8s). When surveying whether identified secreted markers were expressed in CD8s or by the APC line used in high-parameter secreted proteome experiments, we determined that 2/3 of the enriched HLA-E-restricted cytokines were upregulated in at least one E-restricted cluster while 4/7 of the B57-restricted cytokines were also upregulated in at least one B57-restricted cluster (FIGS. 8A-8B).

Lastly, we developed a novel high-throughput and scalable Chi-squared analysis pipeline to identify α, β, and paired αβ families or clonotypes which significantly increased from unstimulated to stimulated conditions in at least one metadata combination. This allowed us to better determine which CD8s belonged to antigen-specific and HLA-restricted TCR family/clonotype subsets. Numerous single and paired chain families were identified, which were then attached as metadata to 10× transcriptome data. Of these, CD8s with TCR clonotypes which were found to significantly increase with antigen stimulation were called as metadata and subset for downstream analysis as an antigen-specific subset.

### Previously Identified High Affinity Cross-Restricted Metaclonotype Found to Significantly Increase Upon Stimulation

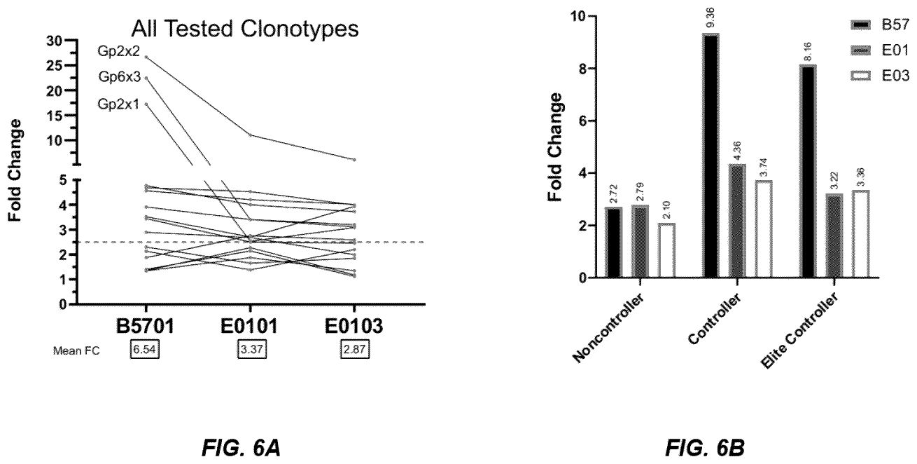

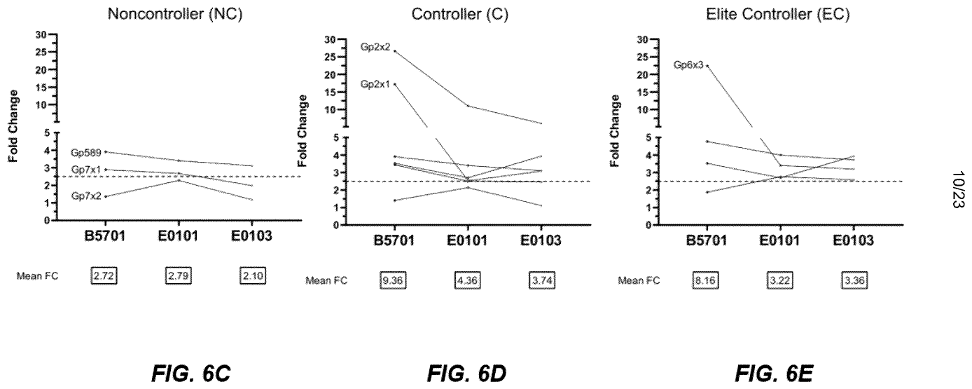

We again identified a TRAV5-TRBV19 cluster of TCRs to be not only tri-restricted but also dominated again by the controller and elite controller. Sequences of TRAV5-containing clonotypes previously identified (Bansal, A. et al. J. Clin. Invest. 2021 131:148979) induced strong NFAT signaling when cloned into a Jurkat NFAT-Luciferase reporter cell line and co-cultured with HLA-E*01:01 or HLA-B*57:01-expressing cell line and pulsed with KF11, demonstrating their functional cross-restriction (FIG. 3) (Bansal, A. et al. J. Clin. Invest. 2021 131:148979). This same cluster of metaclonotype TCRs (TRAV5-TRBV19) was also identified again in the 10× approach in activated/stimulated CD8s from the n=3 PWH, though with far more homogeneous clonality in both their CDR3 clonotypes and αβ family pairs within the controller and elite controller (FIGS. 6A-6E). There were far more cross-restricted clusters observed in the higher depth 10× Tcrdist3 metaclonotype analysis. Still, of particular interest were clusters 1, 3, 4, 5, and 6. In cluster 6, the same TRAV5-TRBV19 cluster previously identified was also enriched again for controller and elite controller TCRs, but this time because PBMC CD8s were included was also found to be highly enriched for controller PBMC TCRs. Cluster 5 was less tightly clustered but also TRAV5-dominated, while clusters 4 and 1 were represented by tri-restricted high proportion clonotypes specific to only the controller or the elite controller, and cluster 3 with mostly non-controller clonotypes.

### IL15 Vaccine Response Signature Unique to Dual Simulated/Activated HLA-B57/E0*01:01-Restricted Elite Controller Cluster

We also wanted to determine if, by subsetting the data by only those significantly increasing αβ clonotype/family TCR CD8s, we could identify markers which differentiated only the most antigen-specific responding T cells from one another by metadata. Initially, upon reclustering, there appeared to be much tighter clustering both in space as well as by metadata when subsetting on significantly increasing clonotypes than by families. This subset clustered much more distinctly by HLA status than in the holistic data, though separation was still mostly driven by control status. Interestingly, when examined more closely, different clonotypes also existed (in most cases) within different clusters, separated by transcriptomic signature. This suggests that these HIV-1 specific CD8s are actually functionally distinct from one another even at the clonotype level and, more importantly, that we can distinguish between them in this analysis.

We next examined the memory subsets enrichment in each HLA-enriched cluster. Metadata assignment of activation was done using CITE-seq expression levels of the activation markers CD69, CD137, and LAMP1, with positivity requiring quantile expression over a cutoff for at least 2/3 of these markers. Similarly, assignment of memory was performed based on expression levels of memory markers CCR7 (RNA expression) and CD45RA (CITE-seq). We determined that KF11-specific CD8s were primarily TEMRA or EM, which was consistent with previous studies on E-CD8s in other pathogens. However, there was no enrichment for a particular memory state by HLA-restriction status, suggesting the same subset has affinity for the same antigen restricted by HLA-B57 or -E. Clusters were found to be distinct by their memory state, HLA restriction, level of control, the stimulation and activation status of CD8s, as well as the number of differentially expressed genes (DEGs) differentiating them from all other clusters by MAST analysis.

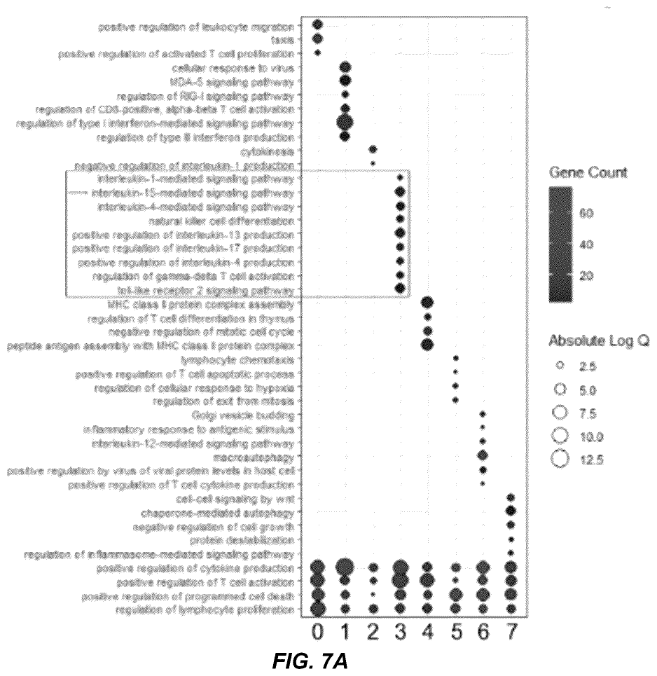

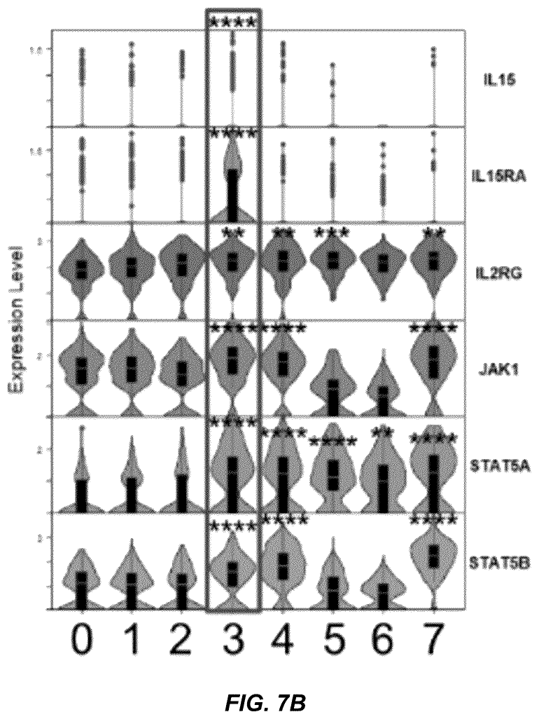

We previously determined that the secreted proteomes of B57-CD8s were functionally distinct from E-CD8s, and this was again found to be the case within this ag-specific subset between B57- and E01-restricted responses. Interestingly, E03-CD8s were not as distinct from B57-CD8s as were E01-CD8s, but rather seemed a subset of both responses. To examine these responses with more granularity, respecting functional distinction arising from differences in memory state and control state as opposed to HLA alone, we used the DEGs identified by MAST between all clusters and used these markers to perform gene ontology analysis (FIG. 7A). When performing this unbiased analysis, we identified only a single cluster enriched for an interleukin-15-mediated signaling pathway signature, cluster 3, or “3 TEMRA E03_B57 Stimulated Activated” cluster after metadata assignment (FIG. 7A). Within this cluster, 6/6 genes mapping to this pathway were identified as upregulated, most interestingly and uniquely as compared to all other ag-specific clusters (FIG. 7B).

### Discussion

Our antigen specific and single cell TCR sequencing of CD8+ T cells obtained from 8 PLW show that TCRs which contain CDR3 α chains belonging to the TRAV5*01_TRBV*19 family can cross-recognize KF11 when presented by HLA-B*57:01, E*01:01, and E*01:03 primarily in controllers and elite controllers, whereas a majority of CD8+ T cells from non-controllers recognize KF11 primarily restricted by HLA-B*57 (TRAV14/DV4-TRBV2). It should be noted that both TRAV5*01_TRBV19*01 as well as TRAV14/DV4 have been previously observed to be specific for KF11 (Yu, X. G. et al. J. Virol. 2007 81:1619-1631). Additionally, the top clonotype pairs previously identified in the same samples utilizing Smart-seq2 based scTCR-seq using tetramer-KF11-based sorting were also identified within our CD3-dim AIM based sorting subset, indicating that these clonotypes are the most antigen-specific and high-affinity (Simons, B. C. et al. J Immunol. 2008 181 (7): 5137-46; Pilkinton, M. A. et al. J. Virol. 2021 95:e02380-20). This metaclonotype was also confirmed when an in-depth single cell based sequencing analysis (10×) of a subset of these participants was performed, but this time also determined to be significantly increasing only in HLA-restricted C and EC CD8s as well as in C/EC PBMC condition CD8s suggesting that this is also a feature of a standard response outside of the co-incubation system. The data suggests that the control of HIV infection, may be partly dependent on the cross-recognition of HIV-1 across HLA-Ia and HLA-E. It is known that HIV-1 downregulates HLA-Ia mediated by Nef and perhaps Vpu, a mechanism which has been well characterized as a means by which it escapes and evades the T cell-mediated immune response (Ende, Z. et al. J. Virol. 2018 92:e01633-17; Apps, R. et al. Cell Host Microbe 2016 19:686-695; Mwimanzi, F. et al. AIDS Lond. Engl. 2020 34:1325-1330). It may well be then that as the founder virus adapts to and downregulates the HLA-Ia of an infected individual, HLA-E expression remains unaffected to present that same epitope to the same subset of CD8+ T cells which would recognize HLA-Ia-restricted HIV-1 epitopes, though the initial original purpose of HIV for leaving HLA-E expressed is likely to evade NK cell clearance (Romero-Martin, L. et al. Front. Immunol. 2022 13:1027855). Whether there are other TCR αβ markers of cross-HLA-Ia/E-restricted responses to other HIV specific and HLA-E restricted epitopes remains to be determined.

A recent study on clonotypes induced by a poorly performing and reduced functionality inducing Ad5 HIV vaccine looked closely at the repertoire of three B*57 controller/long term non progressor (LNTP) vaccinees (Migueles, S. A. et al. Science 2023 382:1270-1276). They described several poorly-elicited TRAV5-TRBV6-1 and TRAV5-TRBV7-9 clonotypes which map to clusters 5 and 6, the “tri-restricted” clusters we identified in ART-naïve controllers and elite controllers by both targeted SMART-seq2 based scTCR-seq and 10×. However, the dominant clonotype in the study found to be elicited was TRAV20-1, which is cluster 1 in the present study, and thereby could be a feature of noncontrollers. Furthermore, this study suggested that the vaccine drove insufficient levels of clonal selection, perhaps eliciting the “wrong” controller-associated antigen-specific and (also seen in our study) tri-restricted efficacious CD8 TCR clonotypes. Additionally, the main finding of the study is that the TCRs elicited in vaccinated participants were low affinity. Our current findings suggest that these low affinity CD8 T cells are not likely to cross recognize epitopes presented by HLA-E thereby potentially negatively impacting vaccine efficacy.

However, despite the observed cross-restriction, which appears to be a viral control-associated phenomenon, when recognizing the same antigen on HLA-B*57 or HLA-E*01:01 or *01:03 these CD8s appear to be functionally and transcriptionally distinct. In our study, RANTES, IP-10, and IL27 were observed to increase in an HLA-E restricted and antigen-specific manner, while more classical CD8 cytokines were observed in the B57-specific response (IFNγ, TNFα, MIG, IL6, MIP-1β, IL2, and IL9). In addition, 2/3 E-CD8 and 4/7 B57-CD8 cytokines were confirmed to be transcribed by the CD8 fraction of the co-culture assays based on scRNA seq data.

Determining whether HLA-restriction is unique to different memory subsets may be important to determining the long-term role of E- vs B57-CD8s in the control of HIV infection. To identify whether HLA-restriction by B57 or E involved unique memory subsets, we also compared the relative numbers of B57-CD8s and E-CD8s that were assigned to each memory subset as metadata. Previous studies have suggested that HCMV-specific E-CD8s display a T cell Effector Memory re-expressing CD45RA or TEMRA (CD45RA+ CCR7−) whereas Mtb-HLAE tetramer specific E-CD8s display predominantly TEMRA or naïve phenotypes (20-30%, 30-40%, respectively), with a comparable central memory population (20%) upon stimulation (Jouand, N. et al. PLoS Pathog. 2018 14:e1007041; Joosten, S. A. et al. J. Immunol. Res. 2016 2016:2695396; Prezzemolo, T. et al. Eur. J. Immunol. 2018 48:293-305). Consistent with prior studies, our data showed that both B57- and E-CD8s were primarily of the EM or TEMRA memory subset, with no difference in proportion of CD8 memory states observed between them. This further suggests that HIV-1 KF11-specific CD8s which can cross-recognize the same antigen between different HLAs but respond differently based on HLA are from the same population and overlapping memory subsets.

Recent work based on bulk RNA-sequencing analysis showed that post SIV vaccination inducing MHC-E-CD8s, an IL15 response uniquely distinguished macaques where establishment of infection was prevented from those in whom it was not (Barrenäs, F. et al. PLoS Pathog. 2021 17:e1009278). Among antigen-specific CD8s specific for KF11, we also observed a cluster of activated, antigen-specific dually-restricted E03/B57-CD8s within an elite controller (EC2) that demonstrated an IL15-signalling pathway transcriptional signature. In the previous study where this IL15 signature was correlated with prevention of SIV infection establishment (Barrenäs, F. et al. PLoS Pathog. 2021 17:e1009278), due to the nature of bulk RNA-seq it was not possible to determine which cell type was responsible for this signaling pathway, nor which cell types were responsible for secretion of IL15. We determined here that it was likely these dually restricted B57- and E-restricted CD8s that were both responding to (IL15RA, various downstream TCR stimulation markers) as well as, to some extent, producing that IL15. Further studies would need to be performed to determine whether this dually restricted IL15 response signature in CD8s is a feature of other elite controllers and tri-restricted clonotypes. Clearly, control in humans and prevention of infection establishment in macaques are not equivalent. Still, this response overlap does raise the question of whether elite control in humans shares characteristics of the infection establishment prevention observed in these macaques. There are almost no cases of spontaneous and persistent clearance of HIV-1 in human patients²³, but it could be that in some people who demonstrate elite control and are long term nonprogressors (LTNPs), some have cleared infection. A general feature of EC long term LTNPs agreed upon in the literature is that they have undetectable plasma viral load for 10 years or more, and it has been proposed that these are the ideal cohort within which to model a functional cure (Tarancon-Diez, L. et al. EBioMedicine 2018 28:15-16; May, M. E. et al. Future Microbiol. 2017 12:1019-1022). It is thought that many EC harbor at most defective replication-incompetent virus, though it has been shown that many are still able to productively infect others (Blankson, J. N. et al. J. Virol. 2007 81:2508-2518; Ho, Y.-C. et al. Cell 2013 155:540-551). Identifying a large enough cohort of these PWH who do not productively transmit would require much more extensive screening of current EC LTNP cohorts either with invasive lymph node biopsies or potentially a more scalable assay involving serotransfer of EC human plasma to humanized mouse models, a viable model which has already been used for this purpose in isolated cases (Salgado, M. et al. J. Virol. 2014 88:3340-3352).

### Methods

Study cohort and HLA-I genotyping: Leukapheresis PBMCs from 8 ART naïve people living with HIV and expressing HLA-B57 were used in this study. These samples were recruited at the University of Vanderbilt, Tennessee. PWH disease status was determined based on viral load of subjects at the time of sample acquisition (Viral load >2000 RNA copies/mL (noncontroller NC), <2000 (controller C), <50 (elite controller EC)). The clinical and HLA typing data is shown in Table 1.

Cell lines: Parental 721.221 or cell line was first knocked out for HLA-E expression at a site in the first exon to generate the 41A3 cell line, before transduction of CD4 and either HLA-B*57:01, HLA-E*01:01, or HLA-E*01:03 to generate the 41A3.CD4. [B57, E01, or E03] cell lines (Bansal, A. et al. J. Clin. Invest. 2021 131:148979). These cell lines were cultured in R-20 (20% FBS in RPMI-1640 supplemented with Penicillin/Streptomycin, L-glutamine, HEPES, and Sodium Pyruvate) and assessed for cell surface HLA expression of the various HLA molecules and CD4 via flow cytometry.

Peptide: HLA-B*57-restricted peptide KF11 (KAFSPEVIPMF, Gag [162-172]) was purchased from Genemed Synthesis, reconstituted in 100% DMSO, and stored at −80° C. until use. HIV-1 Gag, Nef, and Pol overlapping peptide pools were obtained from the NIH AIDS Reagent Program (Catalog number ARP-8117, -12545, and -12438 respectively). Peptide pools were used at 1 μg/mL and single peptide was used at 10 μg/mL, unless otherwise specified.

IFN-γ Enzyme-linked Immunosorbent Spot-forming Assay (ELISPOT): Initial ELISPOT screening of sample IFNG-based release for response rate to previously described HLA-B*57-restricted HIV-1 epitopes was performed, including HLA-B*57-restricted KF11 (KAFSPEVIPMF, Gag [162-172]) as previously described (Bansal, A. et al. J. Clin. Invest. 2021 131:148979; Boppana, S. et al. J. Infect. Dis. 2019 220:1620-1628; Qin, K. et al. J. Virol. 2021 95:e0016021). In brief, cryopreserved samples were thawed in R-10 media (RPMI-1640 base+Streptavidin/Penicillin, L-glutamine, and HEPES), PBMCs allowed to rest at 37° C. O/N. Nitrocellulose plates (MilliporeSigma) were coated with 10 ug/mL α-IFN-γ monoclonal antibody overnight at 4° C. excess uncoated antibody was washed off before wells were blocked with 200 μL R-10 for 2 hours at 37° C. R-10 was discarded and then PBMCs were plated in duplicate wells at 100k cells/well in 100 μL each before antigen was added in 100 μL for a final concentration of 10 μg/mL of single antigen in duplicate wells. PBMCs incubated with no antigen or phytohemagglutinin (PHA) were used as negative and positive controls, respectively. After ˜18-24 hrs, wells were washed and incubated with biotinylated α-IFN-γ antibody for 2 hrs, washed again and incubated with streptavidin-alkaline phosphatase (SA-AP) for 1 hour, then developed with NBT/BCIP substrate for 5-10 minutes. The CTL ImmunoSpot analyzer (version 5) was used to count raw spots/0.1e6 cells which was normalized to spots/1e6 cells (SFU/1e6 cells). A positive response was defined as one which was at least 55 SFU/1e6 cells and 4 times background (DMSO).

Co-culture assay: The dually CD69/137 positive response to HLA-expressing lines pulsed with KF11 was compared to that of the parental line (721. 221ΔE.E) HLA-null line pulsed with KF11 (FIGS. 9A, 9B). The activated population was compared in the CD4/14/16/19/56−, CD8+ CD3-dim population, as CD8+ T cells downregulating CD3 and CD8 coreceptors have been shown to be more antigen-specific and higher avidity (Clutton, G. T. et al. J. Immunol. 2020 204:94.2-94.2; Clutton, G. T. et al. J. Immunol. 2021 206:103.14-103.14; Huang, M. et al. J. Immunol. Methods 2019 472:35-43; Trimble, L. A. et al. J. Virol. 2000 74:7320-7330; Trimble, L. A. et al. Blood 2000 96:1021-1029), and thus are a better indicator of an HLA-restricted antigen specific response in the cell line-dependent coincubation system where nonspecific activation is commonly observed. Those donors who demonstrated a CD69+/137+ response in this subset which was 3 times and significant by Fischer's Exact T test (p-value <0.0001) over the 41A3.CD4 null line response were identified as demonstrating a high-avidity HLA-restricted response to KF11.

Ex vivo activation based single-cell sorting and a sequencing96-well plate scRNA/TCR sequencing: Assays were done as previously described (Bansal, A. et al. J. Clin. Invest. 2021 131:148979) with the following alterations. Initially, indicated condition cell lines (41A3.CD4, 41A3.CD4.B57, 41A3.CD4.E01, 41A3.CD4.E03) were pulsed with KF11 peptide at 10 ug/mL for 2 hrs at 37° C. before peptide was washed off with 1× serum-free media (SFM). CD8+ T cells were then isolated from PBMCs using the StemCell Easy-Sep CD8 Enrichment kit (cat. NC0050243) and co-incubated with peptide-pulsed cell lines as indicated for 18 hrs in the presence of α-CD28 and -49d. Cells were then stained with Aqua LIVE/DEAD stain (Thermo Fisher Scientific), anti-CD3-PacBlue, -CD4-AlexaFluor780, -CD8-AlexaFluor700, -CD14/16/19/56-PerCP-Cy5.5 (dump channel, all separate antibodies), -CD69-APC, -CD137-PE, -CD94-PE-Cy7, and -CD107a-FITC. Single cell lymphocytes were gated on, followed by live cells (Aqua-), CD4/dump−/−, CD8+ CD3-dim, CD69/137+. Cells which fit this gating strategy and met the criteria of having a CD3-dim activated response to the given HLA pulsed with KF11 significantly (and 3×) higher than 41A3.CD4 also pulsed with KF11 were then sorted into a 96-well plate containing the lysis buffer previously described using the Aria II FACS sorter. scTCR and scRNA-seq was performed as previously described (Bansal, A. et al. J. Clin. Invest. 2021 131:148979; Qin, K. et al. J. Virol. 2021 95:e0016021; Files, J. K. et al. J. Virol. 2022 96:e0119122; Currenti, J. et al. Front. Immunol. 2021 12:746986).

10× sample preparation: CD8+ T cells from 3 PWH, 1 from each control status (n=1 NC, C, EC) of the previous n=8 cohort, were co-cultured with CD8+ T cells as described for that set of experiments. These co-cultures were performed in wells of a 24-well plate as per instruction of the Single Cell and Flow Cytometry Core at UAB. After 18 hrs co-incubation, cells were washed and first stained with the Hashing antibodies 1-10 (C0251-C0260) from Biolegend (cat # of C0251 is 394661 for reference to set) per manufacturer instructions post initial titration of both hashtagging antibodies. After washing off, cells were stained using the same panel as previously outlined in addition to the following CITE-seq antibodies with the exact same staining protocol (TotalSeq-C a-CD3, CD4, CD8, TCRαβ, CD56, CD69, CD137, CD107a, PE, APC, FITC, CD45RA, CD45RO, and CCR7). Unactivated (CD69+/137+) CD56-CD8+ cells from the unstimulated (no KF11 loading for 41A3 lines or pulse for PBMCs) as well as both unactivated and activated CD8+ T cells from stimulated conditions were sorted using the BD Symphony S6 cell sorter into a single tube, multiplexed for all 10 samples for one of the 3 PWH per day. Libraries of these cells were then generated on that same day for RNA EXPRESSION (gene expression), TCR, and CITE-seq before these separate hashtagged libraries were then all sequenced on the NovaSeq using an S1 flow cell to cover 1.6 billion reads across the estimated 22,172 cells sorted post manual quality control pre-library generation. Specifically, Cell suspension, 10× barcoded gel beads, and oil were loaded into 10× Chromium™ Single Cell Chip K (PN #1000287). Using Chromium Next GEM Single Cell 5′ v2 Kit (PN #1000263) kit to capture single cells in nanoliter-scale oil droplets by 10× Chromium™ Controller and generate Gel Bead-In-EMulsions (GEMs). The 5′-biased gene expression, TCR and feature barcoding libraries were generated following the instruction. All libraries were sequenced by Illumina Novaseq6000 machine, targeting read pairs 20,000/cell for gene expression and 20,000/cell for TCR libraries and 5000/cell for feature barcoding libraries, and the sequencing cycles consists of 26 bp for read 1, 90 bp for read 2 and 10 bp for i7 and i5.

TCR clustering analysis: Initial clustering analysis was performed as previously described (Bansal, A. et al. J. Clin. Invest. 2021 131:148979). Instead of TCRdist2, however, the newest release TCRdist3 was used for the analysis (Mayer-Blackwell, K. et al. Methods Mol. Biol. Clifton NJ 2022 2574:309-366). As previously described, TCRdist uses the amino acid similarity of Complementarity-determining regions (CDRs) 1, 2, and 3 Initial Hamming distance and distance matrix generation was initially performed using this Python package through R (Reticulate package). After initial distance matrix generation was performed for every clonotype observed in the dataset (with associated metadata), Cytoscape was used to generate clusters. Metadata was added and associated with individual clonotypes based on the metadata sheet generated post TCR pre-processing, and this was used to associate these clonotypes with their HLA-restriction, control, or stimulation state based on previous analysis.

Luminex assay and Analysis: The Luminex Assay was performed using the MagPix instrument running xPONENT® software (Luminex Corporation, Austin, TX, USA) in collaboration with Dr. Davide Botte of the Lund lab. Readouts were analyzed with EMB Millipore's Milliplex Analyst Software. Standard curves and quality control data available upon request. A custom 38-plex human cytokine/chemokine panel was used for this assay (Cat HCYTA-60K-PK38) for the following cytokines: EGF, CCL11, G-CSF, GM-CSF, IFNα2, IFNγ, IL-1α, IL-1β, IL-1RA, IL-2, IL-3, IL-4, IL-5, IL-6, IL-7, IL-8, IL-10, IL-12 (p40), IL-12 (p70), IL-13, IL-15, IL-17A, IL-17E, IL-17F, IL-18, IL-22, IP-10, MCP-1, M-CSF, MIG, MIP-1α, MIP-1β, PDGF-AA, PDGF-AB/BB, RANTES, TNFα, LTA, VEGF-A. The samples assayed were 200 μL supernatants from HLA-null or overexpressed 41A3.CD4 cell lines loaded with either no peptide or KF11 and co-cultured with isolated CD8+ T cells from leukapheresis samples of PLW at various control states. The supernatants were initially spun down to exclude cell debris and then stored at −80° until the time of the assay. Initially, raw data was quality controlled and any cytokine with values outside the standard curves obtained as per manufacturer recommendation were excluded (only IL17-F, which was all below limit of detection). Raw MFI signal values for each sample for each cytokine were compared to the standard curves to generate pg/mL values through the proprietary analysis software. Values for each donor sample were then normalized to the 41A3.CD4-KF11 pulse co-culture condition value of that sample as the optimal negative control for a non HLA-restricted non-allogeneic response. These normalized values were then analyzed initially through Graphpad PRISM for direct pairwise comparisons. All response magnitudes were then visualized through JMP alongside statistically significant pairwise ANOVA comparisons. To analyze these data, a 2-way ANOVA was first performed on both the unstimulated and HLA-KF11 response replicates between HLA-B*57 and -E*01:01 or -E*01:03 as normalized to HLA-null line-KF11 background pg/mL. Those cytokines for which HLA-restriction as a categorical variable explained a significant (p<0.05) amount of the quantitative variation in concentration of the listed cytokine (pg/mL) in the 2-way ANOVA or which demonstrated a significant and substantial increase in only HLA-B*57 or E*01:01/03 conditions were identified. Determinant analysis and canonical clustering were also performed through JMP utilizing available metadata under “Analysis”>“Multivariate methods”>“Discriminant”, with each individual cytokine for all HLA-dependent conditions given as a covariate numerical value and grouped by that condition. Individual cytokines which were shown to demonstrate a significant increase in response between HLA-B57 and either E condition were then represented through PRISM.

Statistical Analysis: Luminex pairwise 2-way ANOVA and t-test significance testing as well as Fisher's exact tests used for flow cytometry analysis were performed using GraphPad PRISM version 8.0 software. Differentially expressed gene (DEG) significance testing was performed using the MAST test through Seurat as described in the package.

### HLA Stabilization Assay

41A3.CD4.E01 HLA-E*01:01 expressing cell line was cultured as described in Methods. 100,000 cells were plated in a 48 well tissue culture plate in R-20 media for 18 hrs at 26° C. Media was washed off and replaced with serum-free media (SFM) containing either 2% DMSO or 100 μg/mL of the indicated peptide before being incubated at 26° C. for 3 hrs. Cells were washed, stained with Fixable Aqua Live/Dead (Invitrogen) and α-HLA*E (clone 3D12, eBioscience), washed again, and fixed with 5% Formalin. Cells were run on the Symphony A3 flow cytometry instrument. All conditions had 3 technical replicates. Median fluorescent intensity (MFI) for all 3 replicates was averaged.

### Peptide Exchange and ELISA

Pre peptide exchange pHLA-E (VL9 “J” VMAPJTLVL peptide loaded) monomer was generously provided by the Emory Tetramer Facility Peptide exchange was performed in a modified form from what has described (Walters, L. C. et al. Eur. J. Immunol. 2020 50:2075-2091; Yang, H. et al. Sci. Immunol. 2023 8: eabl8881; Walters, L. C. et al. Nat. Commun. 2018 9:3137; Toebes, M. et al. Curr. Protoc. Immunol. 2009 87). Following various optimization experiments, the protocol for this assay was as follows. Initially, ELISA high-bind plates were coated with 10 μg/mL coating with purified α-HLAE antibody (3D12) overnight at 4° C. After initial overnight coating at 4° C., another overnight blocking with 2% IgG BSA (cat #12-662-550ML) was performed. The following day, peptide exchange was performed in wells of a V-bottom plate. Each 125 μL reaction contained 121.5 μL of peptide exchange buffer, 1.5 μL stock monomer (3 μg 7MT2, stored at 80° C.), and 2 μL query peptide (200 μg). Peptide exchange buffer stock generated using 1 mL 1M Tris-HCl (pH 8), 842.64 mg L-arginine monohydrochloride, 40 μL 0.5M EDTA, 15.4 mg reduced glutathione, 3 mg oxidized glutathione, 8.1 mL ddH2O, and one peptidase inhibitor complete EDTA-free tablet (respectively, cat #AAJ22638AE, 11-101-2479, MT-46034CI, AC120000050, MP021511935, N/A, and 11873580001). Each reaction was gently mixed with a pipette and then UV-irradiated (365 nm) for 3 hrs at RT. The coated/blocked ELISA plate was then washed as previously described⁹ and incubated with 50 μL (1:100) of peptide-exchanged reaction mix for 1 hr. This was carried out at 37° C. as previously described to enhance binding (Toebes, M. et al. Curr. Protoc. Immunol. 2009 87). After washing, 1:500 50 μL 1° ab (α-β2M cat #R0202-1D) was incubated on the plate for 30 mins at 4° C., then after another round of washing 1:500 50 μL 2° ab (α-Rabbit-HRP cat #NV7160) for 15 mins at RT in the dark. Finally, plate was washed again and 100 μL TMB substrate (cat #PI37574) was added for 10 mins (or until sufficient development) before adding 100 μL ELISA Stop soln (cat #PIN600). Corrected A450 values (-background A650) were transformed as follows. TMB substrate and stop solution was added by row, and so corrected A450 values for each row were normalized separately between 0 (7MT2 (-control) no peptide condition) and 1 (A*02 VL9 (+control) peptide exchange) with the following calculation

$$\frac{query - ( 7 MT 2 unloaded ]}{A * 02 VL 9 - 7 MT 2 unloaded} .$$

When mentioned, averaged values were calculated across these 4 technical replicate normalized values.

### 10× Analysis Pipeline

10× Demultiplexing: Initially, 3 sets of paired reads FASTA files were obtained for the 3 types of data (RNA EXPRESSION, TCR, and CITE-seq/multiplex hashtag antibody feature barcodes (FB)) for each multiplexed donor for a total of 18 files and 112 GB of gzipped data. Initial de-multiplexing was performed on these 3 donor multiplexed data using the RNA EXPRESSION and hashtags of the FB FASTA files adopting a modified version of the strategy described here so as to in the most unbiased manner use the 5′ Biolegend hashtagging oligos with the 3′ CMO Biolegend demultiplexing pipeline to assign cells to different samples within each donor. A hashing_demux_HMO-set.csv targets file as well as a demultiplex config file were used for this purpose. After initial QC of the number of cells and reads returned for every sample, it became apparent that the elite controller PBMC-KF11 stimulated condition was either not sorted or not amplified. Bam files generated in the demultiplexing process were then converted back to FASTA within donor sample folders under condition folders and TCR and CITE-seq data was then extracted using the cell IDs assigned to each sample from demultiplexing using cellranger multi and a new config file for each sample within each donor directory. This resulted in 87 unique FASTA files, 3 each RNA EXPRESSION, TCR, and FB for the 10 conditions per donor sample under donor/condition directory folder, then used for downstream Seurat analysis.

Initial pre-processing: In-depth Rmarkdown of this process is available upon request including scripts used to generate various figures present in the manuscript as well as to perform pre-processing of data. In brief, a loop was used based on the filestructure naming scheme and both patient as well as condition names to individually construct Seurat objects using CreateSeuratObject, then assigning metadata to each individual object based on the condition and patient names of the filestructure. Initially, RNA EXPRESSION was first imported under the “RNA” assay object, after which FB (containing the CITE-seq expression data) was imported into the “Protein” assay object. Each 10 (or 9 for the elite controller where PBMC-KF11 data was not present) condition objects were then added to SeuratLists by donor within the loop. Metadata was then assigned using another loop based on the naming scheme of these Seurat objects inside their SeuratLists for HLA, donor, control status, and other relevant metadata.

TCR metadata pre-processing: TCR data was processed similarly, but instead of being used to create Seurat objects, individual “filtered_contig_annotations.csv” files output from cellranger under each folder in the filestructure were individually imported. MixCR format was, as always, incompatible with all downstream analysis due to data being separated into rows based on TCR α or β chain rather than by cell, so based on the barcode ID, these were combined into a single row with new column names by cell. This data was then analyzed individually within that sample for proportions of each chain or paired chain as well as CDR3 chain or paired clonotype before then being attached as new metadata columns to the relevant Seurat object within its SeuratList. Unique prefixes were then applied for each individual cell by patient_condition combination. TCR meta-analysis was performed by extracting this metadata (both TCR and other metadata such as stimulation status, hla condition, and control status from each object and combining all of it into a single data table as “combined_metadata.csv”. Proportions of each TCR αβ individual or paired chain CDR3 clonotype or family for every combination of metadata (control, HLA, stimulation, all or sub-combination of any of these) was calculated across all cells matching that metadata combination. Counts of each of those single or paired chain family or CDR3 sequences were then also tabulated by all combinations. Delta proportions for each combination from DMSO to KF11 stimulation conditions were then calculated and a novel Chi-squared test was performed for every single or paired chain between stimulation conditions to determine whether a significant delta increase or decrease was observed. These results were condensed to only unique chain families or CDR3 sequences (single or paired) as separate tabs of an output excel spreadsheet, separated by α, β, or paired αβ. A new sheet containing curated significantly increasing TCRs from this output was reformatted for TCRdist3 analysis as previously. Family and clonotype analysis were performed in separate steps. Clonotype IDs as determined across all data (not within each condition) were then assigned to the downstream merged SeuratObject for all samples.

RNA expression pre-processing and QC: Initial pre-processing of gene-expression analysis was carried out by first iterating through each Seurat object within each list and Log normalizing and scaling the data using the RNA expression data before finding variable features and filtering out any cells which had mtRNA %<5 and nFeature_RNA >200. QC metrics plots for these as well as nCountRNA were generated after this step to visualize distribution of data within samples and ensure cutoffs were successfully executed with the cowplot package. Because each individual sample was its own either control status or HLA condition and there was virtually no batch effect (all library creation and sequencing was performed immediately post sorting and within 1 week of each other), integration was not pursued as a method to generate a single unified Seurat object containing all data across all samples. Within each patient SeuratList, all condition Seurat objects were merged into a single patient Seurat object (3 objects, 1 per patient). These objects were then merged to form a single merged Seurat object for all samples containing all metadata, to which clonotype id information was applied to each cell based on the cdr3_a_b_aa pair sequence. nFeature and nCount upper limit cutoffs were at this point applied as QC for doublets, with the cells possessing top 2% quantile of each filtered out as outliers. As an additional quality control, DoubletFinder was used to detect doublets based on simulated data comparisons, with the optimal pK of 0.001 and pN of 0.25 used as determined by highest average BCReal value. To determine the number of dimensions on which to run nNeighbors and generate UMAPs, an ElbowPlot was used, after which the first 15 dimensions were found to be optimal. The Clustree package was then used to determine the optimal resolution to generate Seurat clusters such that indeterminant cross-transfer was minimized (to prevent over-clustering) (Zappia, L. & Oshlack, A. GigaScience 2018 7:giy083).

CITE-seq expression pre-processing into metadata: Utilizing the CITE-seq markers for CD45RA and the mRNA expression level of CCR7 (CITE-seq staining of CCR7 was suboptimal and did not match mRNA expression) across Seurat clusters, clusters were subset into naïve (N), effector memory (EM), terminal effector memory re-expressing RA (TEMRA), and central memory (CM) with the same “gating strategy” based on expression level above or below a percentile cutoff based on the same gating strategy by which these states would be determined in conventional flow cytometry (CCR7_, CD45RA_+/+, −/−, +/−, −/+ denoting N, EM, CM, or TEMRA respectively). A similar methodology was used for determination of activation status, wherein triple positivity of CITE-seq CD69, CD137, and CD107a expression above an expression threshold cutoff was used to denote “activated” or “unactivated” status of a given cell. Metadata for HLA restriction, control status, stimulation status, and derived activation and memory state status was used to denote cluster metadata status based on relative. In brief, the number of cells corresponding to each state was output into a table format and converted into Z-scores within a heatmap scaled by cluster (as opposed to scaled to all clusters). This Z-score was then used to determine the metadata status of this cluster. For example, if Z-scores for number of B57- and PBMC-metadata CD8s were within 0.6 of one another but higher than 0.6 over all other HLA statuses for a given cluster, this cluster would be denoted B57_PBMC, with the same process used for all other metadata types in each cluster. This ensured the absence of bias, similar to the expression level cutoff.

TABLE 1
Cohort demographics and clinical.

| | | | | | | | KF11 ELISPOT | | | |
|---|---|---|---|---|---|---|---|---|---|---|
| | Alternate | Viral Load | | | | | IFNγ | | | |
| Status | ID | (cp/mL) | HLA-A1 | HLA-A2 | HLA-B1 | HLA-B2 | SFU/1e6 cells | Luminex | SmartSeq2 | 10X |
| C 1 | Cl10004 | 253 | A*03:01 | A*30:02 | B*07:02 | B*57 | 2400 | Used | Used | Used |
| C 2 | Cl10060 | 722 | A*01:01 | A*33:01 | B*42:01 | B*57 | 415 | Used | Used | |
| C 3 | Cl10067 | 215 | A*30:02 | A*33:01 | B*13:02 | B*57 | 610 | Used | Used | |
| C 4 | Cl20018 | 1886 | A*02:01 | A*26:01 | B*40:01 | B*57 | 1520 | Used | Used | |
| EC 1 | Cl10071 | 49 | A*01:01 | A*66:02 | B*8:01 | B*57 | 730 | Used | Used | |
| EC 2 | Cl10074 | 49 | A*01:01 | A*26:01 | B*8:01 | B*57 | 770 | Used | Used | Used |
| NC 1 | Cl10027 | 4827 | A*01:01 | A*02:01 | B*8:01 | B*57 | 714 | Used | Used | |
| NC 2 | Cl10076 | 17693 | A*02:01 | A*30:02 | B*35:01 | B*57 | Not tested | Used | Used | Used |
| PWO 1 | | 0 | A*30:01 | A*68:02 | B*42:01 | B*57 | Not tested | | | |
| EC 3 | | 47 | A*02:01 | A*30:02 | B*07:02 | B*57 | 2737 | | | |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for identifying a variable chain receptor (VCR) family/clonotype subset for an antigen, the method comprising:
(a) isolating at least one type of lymphocyte from a plurality of subjects having a disease associated with the antigen, where the plurality of subjects are classified into two or more disease conditions based on detectable levels of the antigen in the plurality of subjects;
(b) culturing the at least one type of lymphocyte with a plurality of disease-appropriate cell lines to create cell populations representing each of the two or more disease conditions, to provide unstimulated cells for each of the two or more disease conditions;
(c) culturing the at least one type of lymphocyte with the plurality of disease-appropriate cell lines loaded with antigen to provide stimulated conditions for each of the two or more disease conditions;
(d) conducting single-cell RNA sequencing (scRNA-seq) and single-cell TCR sequencing (scTCR-seq) or single-cell BCR sequencing (scBCR-seq) on each of lymphocyte from steps (b) and (c) to identify the count and sequence of VCRs in each lymphocyte across all conditions;
(e) identifying families or clonotypes for lymphocytes that had a statistically significant change in count and frequency between stimulated and unstimulated conditions; and
(f) comparing each of the two or more disease conditions to identify antigen-specific family/clonotype subsets; and
(g) cloning identified VCRs into a cell based reporter system to test whether, in the presence of the specific antigen, disease condition, relevant stimulant, or any combination thereof, the VCRs demonstrate functional avidity.

2. The method of claim 1, wherein the VCR comprises a T cell receptor (TCR), wherein the at least one type of leukocyte comprises a CD8+ T cell, wherein the two or more disease conditions comprise two or more human leukocyte antigen (HLA) conditions, wherein the family/clonotype subset comprises an HLA-TCR family/clonotype subset, wherein the plurality of disease-appropriate cell lines comprise HLA-expressing cell lines, wherein scRNA-seq and scTCR-seq are conducted in step (d), wherein the family/clonotype subset comprises TCR α, β, paired αβ families or clonotypes, and wherein the cell based reporter system comprises a Nuclear Factor of Activated T-cells (NFAT)-reporter system.

3. The method of claim 1, wherein the VCR comprises a B cell receptor (BCR), wherein the at least one type of leukocyte comprises a B cell, wherein scRNA-seq and scBCR-seq are conducted in step (d), wherein antigen-specific BCR heavy and light chain sequences are transfected into BCR-null expression systems, isolated, and purified; and wherein the cell based reporter system comprises ELISA, multimer detection, surface plasmon resonance (SPR) or any combination thereof.

4. The method of claim 2, further comprising immunoassaying CD8+ T cells identified in step (e) for expression levels of one or more cytokines and growth factors selected from the group consisting of EGF, CCL11, G-CSF, GM-CSF, IFNα2, IFNγ, IL-1α, IL-1β, IL-1RA, IL-2, IL-3, IL-4, IL-5, IL-6, IL-7, IL-8, IL-10, IL-12 (p40), IL-12 (p70), IL-13, IL-15, IL-17A, IL-17E, IL-17F, IL-18, IL-22, IP-10, MCP-1, M-CSF, MIG, MIP-1α, MIP-1β, PDGF-AA, PDGF-AB/BB, RANTES, TNFα, LTA, VEGF-A.

5. The method of claim 4, wherein the immunoassay is a high-parameter Luminex assay.

6. The method of claim 2, further comprising conducting a CITE-seq (cellular indexing of transcriptomes and epitopes) assay on the CD8+ T cells identified in step (e).

7. The method of claim 2, wherein step (f) comprises identifying HLA-restricted TCR family/clonotype subsets that are the most antigen-specific with the highest difference in unstimulated to stimulated conditions.

8. The method of claim 2, wherein the antigen comprises a viral antigen.

9. The method of claim 8, wherein the viral antigen comprises a human immunodeficiency virus (HIV) antigen and wherein step (a) comprises obtaining CD8+ T cells from each of i) an ART-naïve elite controller (EC) subject, ii) an ART-naïve controller (C) subject, and an ART-naïve noncontroller (NC) subject.

10. The method of claim 9, wherein the EC subject has a plasma viral load less than 200, the C subject has a plasma viral load from 20 to 2000, and the NC subject has a plasma viral load greater than 2000.

11. An antigen-specific TCR associated with a positive disease outcome identified through the method of claim 1.

12. A modified allogenic T cell transduced with the antigen-specific TCR of claim 11.

13. A method of treating a subject having a viral infection or cancer, comprising administering to a subject the modified allogeneic T cell of claim 12.

14. A B cell transduced with the antigen-specific BCR of claim 3.

15. An antibody produced by the B cell of claim 14.

16. A method of treating a subject having a viral infection or cancer, comprising administering to a subject the antigen-specific antibody of claim 15.

17. A database of VCRs associated with positive disease outcome produced by conducting the method of claim 1 a plurality of times and storing data obtained therefrom in a computer readable medium.

18. A method for determining effectiveness of a vaccine or drug treatment against a disease, wherein VCRs associated with positive disease outcome are present in the database of claim 17, the method comprising administering the vaccine or drug treatment to a plurality of subjects and comparing data from samples from the subjects to the database to determine presence or absence of the VCRs associated with positive disease outcome in the subjects, where presence of the VCRs indicates the vaccine or drug treatment is effective.

19. A method for determining effectiveness of a treatment for a disease in a subject, wherein VCRs associated with positive disease outcome are present in the database of claim 17, the method comprising comparing data from a sample the subject to the database to determine presence or absence of the VCRs associated with positive disease outcome in the subject, where presence of the VCRs indicates the treatment is effective.

20. A reagent for use in an immunoassay, the reagent comprising an antibody according to claim 15 conjugated to a fluorophore.
